(19) United States
(12) Patent Application Publication
(10) Pub. No.: US 2023/0313271 A1
(43) Pub. Date: Oct. 5, 2023

(54) MACHINE-LEARNING MODELS FOR DETECTING AND ADJUSTING VALUES FOR NUCLEOTIDE METHYLATION LEVELS

(71) Applicants: Illumina, Inc., San Diego, CA (US); Illumina Cambridge Limited, Cambridge (GB)

(72) Inventors: Steven Norberg, La Mesa, CA (US); Luis Fernando Camarillo Guerrero, Cambridge (GB); Colin Brown, San Diego, CA (US); Andrea Manzo, San Diego, CA (US); Sarah E. Shultzaberger, San Diego, CA (US); Michael Eberle, Oceanside, CA (US); Sepideh Almasi, Los Gatos, CA (US); Suzanne Rohrback, San Diego, CA (US); Pascale Mathonet, Cambridge (GB); Egor Dolzhenko, San Diego, CA (US)

(21) Appl. No.: 18/172,821

(22) Filed: Feb. 22, 2023

Related U.S. Application Data
(60) Provisional application No. 63/268,550, filed on Feb. 25, 2022.

(51) Int. Cl. C12Q 1/6809; G16C 20/70

(52) U.S. Cl. CPC C12Q 1/6809; G16C 20/70

(57) ABSTRACT

This disclosure describes methods, non-transitory computer readable media, and systems that can use a machine-learning to determine factors or scores indicating an error level with which a given methylation assay detects methylation of cytosine bases. For instance, the disclosed systems use a machine-learning model to generate a bias score indicating a degree to which a given methylation assay errs in detecting cytosine methylation when specific sequence contexts surround such cytosines compared to other sequence contexts. The machine-learning model may take various forms of models, including a decision-tree model, a neural network, or a combination of a decision-tree model and a neural network. In some cases, the disclosed system combines or uses bias scores from multiple machine-learning models to generate a consensus bias score.

### CROSS-REFERENCE TO RELATED APPLICATIONS

The present application claims the benefit of, and priority to, U.S. Provisional Application No. 63/268,550, entitled “MACHINE-LEARNING MODELS FOR DETECTING AND ADJUSTING VALUES FOR NUCLEOTIDE METHYLATION LEVELS,” filed on Feb. 25, 2022. The aforementioned application is hereby incorporated by reference in its entirety.

### BACKGROUND

In recent years, biotechnology firms and research institutions have improved hardware and software for both detecting methylation of cytosine bases at particular genomic regions (e.g., regions encoding or promoting genes) and detecting methylation of larger nucleotide fragments or whole genomes of a sample. For instance, existing sequencing systems can use sequencing devices and corresponding sequencing-data-analysis software to identify when a methyl or hydroxymethyl grouphas been added to a cytosine base-typically part of a cytosine-guanine-dinucleotide pair in a 5′-C-phosphate-G-3′ (CpG) configuration. For example, existing sequencing systems can detect methylated CpGs by (i) enzymatically converting methylated or unmethylated cytosine bases at CpG sites from a sample nucleotide fragment into uracil bases (e.g., dihydrouracil); (ii) determining base calls of nucleotide reads for the sample using a sequencing device, where the sequencing device detects the uracil bases as thymine bases during polymerase chain reaction (PCR) amplification; and (iii) comparing the base calls from the nucleotide reads to a reference genome. Based on the comparison of nucleotide reads from the sample to a reference genome, existing sequencing systems can identify thymine bases from the nucleotide reads that do not match cytosine bases at CpG sites within the reference genome and thereby detect methylated CpG sites in a sample nucleotide fragment.

To convert cytosine to uracil, in some cases, existing methylation assays use bisulfite as an enzyme, whereas other methylation assays use a non-bisulfite enzyme. For instance, Tet-assisted pyridine borane sequencing (TAPS) uses a TET enzyme for a methylation assay, as described by Yibin Liu et al., “Bisulfite-free Direct Detection of 5-Methylcystosine and 5-Hydroxymethylcystosine at Base Resolution,” 36 Nature Biotechnology 424-29 (2019). While bisulfite can be more reliable as a conversion enzyme, bisulfite can also adversely affect other components of a nucleotide fragment for sequencing.

By performing methylation assays using various enzymes or approaches, existing sequencing systems can detect cytosine methylation for various diagnostic or therapeutic purposes. For example, some existing sequencing systems can perform a methylation assay to determine methylation levels of CpG islands that span gene promoters or exons and thereby use CpG methylation detection as an early biomarker of cancer. Existing methylation assays can similarly be used to determine methylation levels of certain genomic regions for genes relevant to other diseases, such as certain autoimmune, neurological, and psychiatric disorders. In addition to using methylation assays to detect disorders, in some cases, existing sequencing systems perform a methylation assay to determine methylation levels of promoter regions and determine how such methylation affects regulation and expression of genes corresponding to promoter regions.

Despite recent improvements to methylation assays, existing sequencing systems often generate inaccurate methylation measurements for cytosine bases. For example, existing methylation assays often generate beta values or M values indicating a level of cytosine methylation at particular genomic coordinates or regions. But such beta values or M values can misrepresent actual levels of cytosine methylation in a deoxyribonucleic acid (DNA) fragment. As described further below, existing methylation assays can determine beta values or M values for cytosine bases at specific genomic sites with accuracies that vary wildly.

Because existing sequencing systems execute methylation assays that are inaccurate or otherwise unreliable, existing systems may re-run methylation assays on multiple copies of DNA fragments from a sample or run different types of methylation assays to determine more reliable beta values or M values for consensus. But such re-execution of methylation assays or use of different methylation-assay types can consume valuable computing resources on both specialized sequencing devices and computing devices executing sequencing-data-analysis software-thereby performing redundant analyses and performing time-intensive-computer processing on such computing devices, where sequencing runs alone can consume between approximately four to fifty-five hours on specialized sequencing devices. Despite the importance and extreme variability of such methylation assays, the technical cause of some of existing methylation assays’ variation and inaccuracies have been unclear and puzzling prior to this disclosure.

### SUMMARY

This disclosure describes one or more embodiments of systems, methods, and non-transitory computer readable storage media that solve one or more of the problems described above or provide other advantages over the art. In particular, the disclosed system uses a machine-learning to determine factors or scores indicating an error level with which a given methylation assay detects methylation of cytosine bases. For instance, the disclosed systems use a machine-learning model to generate a bias score indicating a degree to which a given methylation assay errs in detecting cytosine methylation when specific sequence contexts surround such cytosines compared to other sequence contexts. The machine-learning model may take various forms of models, including a decision-tree model, a neural network, or a combination of a decision-tree model and a neural network. In some cases, the disclosed system combines or uses bias scores from multiple machine-learning models to generate a consensus bias score.

To illustrate but one embodiment, in some cases, the disclosed system identifies, for a given methylation assay, a methylation-level value indicating a level of methylation of a cytosine base within a sample nucleotide sequence. The disclosed system further uses a methylation-bias-adjustment-machine-learning model to determine a bias score for a contextual sequence flanking the cytosine base. Such a bias score may indicate a degree to which the given methylation assay errs in detecting methylation of the cytosine base when flanked by the contextual sequence. Based on the bias score, the disclosed system can adjust the methylation-level value output by the given methylation assay. Also based on the bias score, the disclosed system can additionally or alternatively identify a genomic coordinate or genomic region for the cytosine base subject to a bias of the given methylation assay.

### BRIEF DESCRIPTION OF THE DRAWINGS

The detailed description refers to the drawings briefly described below.

FIG. 1 illustrates a computing-system environment in which a bias-adjusted-methylation-assay system can operate in accordance with one or more embodiments of the present disclosure.

FIG. 2 illustrates a schematic diagram of the bias-adjusted-methylation-assay system utilizing a machine-learning model to determine bias scores of a methylation assay for a specific contextual sequence of a target cytosine base and adjusting a methylation-level value for the target cytosine base from the methylation assay in accordance with one or more embodiments of the present disclosure.

FIGS. 3A-3C illustrate schematic diagrams of the bias-adjusted-methylation-assay system utilizing different embodiments of a methylation-bias-adjustment-machine-learning model to determine bias scores of a methylation assay or predicted methylation-level values for a specific contextual sequence of a target cytosine base in accordance with one or more embodiments of the present disclosure.

FIGS. 4A-4C illustrate schematic diagrams of the bias-adjusted-methylation-assay system training different embodiments of a methylation-bias-adjustment-machine-learning model to determine bias scores or predicted methylation-level values in accordance with one or more embodiments of the present disclosure.

FIG. 5 illustrates a graphic indicating contribution metrics of different nucleobase classes at different contextual-sequence positions contributing to predicted methylation-level values in accordance with one or more embodiments of the present disclosure.

FIGS. 6A-6H depict graphics indicating degrees to which nucleobase-class changes at different contextual-sequence positions affect bias scores in accordance with one or more embodiments of the present disclosure.

FIG. 7 illustrates a graph showing a percentage of CpG sites for which a methylation-bias-adjustment-machine-learning model correctly determines predicted methylation-level values based on length of contextual sequence flanking a cytosine base in accordance with one or more embodiments of the present disclosure.

FIGS. 8A-8C depict graphs showing methylation-level values from a given methylation assay and corresponding bias scores determined by a methylation-bias-adjustment-machine-learning model in accordance with one or more embodiments of the present disclosure.

FIG. 9 illustrate a series of acts for utilizing a machine-learning model to determine bias scores of a methylation assay for a specific contextual sequence of a target cytosine base and adjusting a methylation-level value for the target cytosine base from the methylation assay in accordance with one or more embodiments of the present disclosure.

FIG. 10 illustrates a series of acts for training a methylation-bias-adjustment-machine-learning model to determine predicted methylation-level values for specific contextual sequences in accordance with one or more embodiments of the present disclosure.

FIG. 11 illustrates a series of acts for training a methylation-bias-adjustment-machine-learning model to determine bias scores for specific contextual sequences in accordance with one or more embodiments of the present disclosure.

FIG. 12 illustrates a block diagram of an example computing device in accordance with one or more embodiments of the present disclosure.

### DETAILED DESCRIPTION

This disclosure describes one or more embodiments of a bias-adjusted-methylation-assay system that uses a machine-learning model to determine factors indicating an error level with which a methylation assay detects methylation of cytosine bases. For instance, the bias-adjusted-methylation-assay system can use a methylation-bias-adjustment-machine-learning model to generate a bias score indicating a degree to which a given methylation assay errs in detecting cytosine methylation when such cytosines are surrounded by specific context sequences. Such a contextual sequence may include upstream and downstream nucleobases flanking or surrounding a target cytosine. Based on bias scores form the machine-learning model, in some cases, the bias-adjusted-methylation-assay system adjusts methylation-level values generated by a given metalation assay. The methylation-bias-adjustment-machine-learning model may take various forms of neural networks or other machine learning, including a random-forest model, a convolutional neural network (CNN), or a combination of a random-forest model and CNN. In some cases, the bias-adjusted-methylation-assay system averages or otherwise combines bias scores from multiple machine-learning models to generate a consensus bias score that reduces the inaccuracies of a given machine-learning model.

As suggested above, in some cases, the bias-adjusted-methylation-assay system identifies, for a given methylation assay, methylation-level values indicating levels of methylation of cytosine bases within a sample nucleotide sequence. The bias-adjusted-methylation-assay system may, for instance, receive methylation-level values determined by the given methylation assay or execute the given methylation assay on one or more computing devices to determine the methylation-level values. The bias-adjusted-methylation-assay system further uses a methylation-bias-adjustment-machine-learning model to determine bias scores for contextual sequences respectively flanking the cytosine bases. Based on the bias scores, the disclosed system can adjust the methylation-level values output by the given methylation assay for the cytosine bases. Likewise, based on the bias scores, the bias-adjusted-methylation-assay system can identify a genomic coordinate or region for the cytosine bases subject to a bias of the given methylation assay.

The methylation-bias-adjustment-machine-learning model can take various forms and be used to determine a bias score in a variety of ways. As an example, the methylation-bias-adjustment-machine-learning model may directly or indirectly generate a bias score. In some embodiments, the bias-adjusted-methylation-assay system generates a predicted methylation-level value based on a dataset representing a contextual sequence flanking a target cytosine base. To determine a corresponding bias score, the bias-adjusted-methylation-assay system can subsequently determine a value difference between the predicted methylation-level value from the methylation-bias-adjustment-machine-learning model and an expected methylation-level value determined by a given methylation assay for a synthetically methylated cytosine base. By contrast, in some cases, the methylation-bias-adjustment-machine-learning model generates the bias score based on a dataset representing the contextual sequence flanking a target cytosine base.

To compensate for the potentially variable performance or accuracy of a given machine-learning model, in some cases, the bias-adjusted-methylation-assay system uses different machine-learning models to determine a consensus bias score. For instance, the bias-adjusted-methylation-assay system can use a first methylation-bias-adjustment-machine-learning model to determine a first bias score for a contextual sequence flanking a target cytosine base and a second methylation-bias-adjustment-machine-learning model to determine a second bias score for the contextual sequence. The bias-adjusted-methylation-assay system can subsequently determine a composite bias score for the contextual sequence-based on the first bias score and the second bias score-and then adjust a methylation-level value for the target cytosine base based on the composite bias score.

After determining a bias score, the bias-adjusted-methylation-assay system can use the bias score for a variety of applications. For instance, the bias-adjusted-methylation-assay system can identify a genomic coordinate or region comprising one or more target cytosine bases that are subject to a given methylation assay’s bias in detecting methylation levels. Such a genomic coordinate or region may correspond to a gene or promoter region for a gene relevant to a genetic diagnosis or genetic predisposition, such as a gene relevant to a type of cancer, autoimmune disease, neurological disease, or other disease. Additionally, or alternatively, the bias-adjusted-methylation-assay system can adjust a methylation-level value for a target cytosine base at a genomic coordinate or withing a genomic region corresponding to such a gene or promoter region.

In addition or in the alternative to adjusting methylation-level values, in some cases, the bias-adjusted-methylation-assay system generates graphics that indicate changes to bias scores specific to contextual sequences flanking target cytosine bases. For instance, in some cases, the bias-adjusted-methylation-assay system generates data for a graphic indicating degrees to which nucleobase-class changes at different positions within a contextual sequences affect bias scores. Additionally, or alternatively, the bias-adjusted-methylation-assay system generates data for a graphic indicating contribution metrics for different nucleobase classes at different positions within contextual sequences contributing to predicted methylation-level values.

To train a methylation-bias-adjustment-machine-learning model, the bias-adjusted-methylation-assay system can take a variety of approaches for training iterations. In some cases, for instance, the methylation-bias-adjustment-machine-learning model determines predicted methylation-level values for specific contextual sequences and modifies parameters of the methylation-bias-adjustment-machine-learning model based on a comparison of the predicted methylation-level values with expected methylation-level values (e.g., via a loss function) for a given methylation assay. Additionally, or alternatively, the bias-adjusted-methylation-assay system determines observed methylation-level values for synthetically methylated cytosine bases from a given methylation assay; determines predicted bias scores for contextual sequences flanking the synthetically methylated cytosine bases; and further modifies parameters of the methylation-bias-adjustment-machine-learning model based on comparisons of the predicted bias scores and expected bias scores (for the contextual sequence) derived from the observed methylation-level values.

As indicated above, the bias-adjusted-methylation-assay system provides several technical advantages relative to existing sequencing systems, such as by improving the accuracy and diagnostic applications of methylation assays. For instance, in some embodiments, the bias-adjusted-methylation-assay system improves the accuracy of detecting methylation levels of CpG sites or other cytosine bases within a sample nucleotide sequence. As suggested above, some existing sequencing systems generate inaccurate methylation-level values (e.g., beta values, M values) that misrepresent actual methylation of particular CpG sites or other cytosine bases. As discovered by the inventors of this disclosure, however, when known CpGs in oligonucleotides are synthetically methylated and processed through existing methylation assays, existing methylation assays generate beta or M values that vary from expected beta or M values for the artificially methylated oligonucleotides. Despite having a priori known methylation levels for such synthetically methylated oligonucleotides (e.g., approximately 100% CpG methylation), existing methylation assays generate beta or M values (e.g., 70% at some CpG sites and 5% at other CpG sites) that vary considerably from the expected beta or M values (e.g., approximately 100%). Beta and M values can likewise vary for genomic regions known to be naturally methylated.

In contrast to such existing methylation assays, the bias-adjusted-methylation-assay system uses a unique methylation-bias-adjustment-machine-learning model to generate a bias score indicating a degree to which a given methylation assay errs in detecting cytosine methylation-when specific sequence contexts surround such cytosines. Accordingly, the bias-adjusted-methylation-assay system generates a contextual-sequence-specific bias score that no previous assay or computing device has generated. By determining a contextual-sequence-specific bias score indicating a degree to which a given methylation assay errs in measuring methylation, in some cases, the bias-adjusted-methylation-assay system corrects or otherwise adjusts a methylation-level value for a target cytosine base (e.g., at a particular genomic coordinate or region) generated by the given methylation assay. Indeed, the bias-adjusted-methylation-assay system can generate contextual-sequence-specific bias scores that correct for failed enzymatic methylation, failed or inaccurate imagining on a sequencing device, or other mechanical or computational errors that hinder existing methylation assays. As explained below, in some cases, the bias-adjusted-methylation-assay system uses a first-of-its-kind methylation-bias-adjustment-machine-learning model comprising network layers that are uniquely structured to generate predicted methylation-level values (or facilitate bias scores) to improve the accuracy of methylation-level values for a given methylation assay.

In addition to improved methylation-level values for a given methylation assay, in certain implementations, the bias-adjusted-methylation-assay system improves the accuracy and efficiency of determining genomic coordinates or genomic regions with methylated cytosine bases subject to biased detection from existing methylation assays. As noted above, some existing sequencing systems generate inaccurate methylation-level values (e.g., beta values, M values) for genomic coordinates or regions with CpG sites (e.g., CpG islands) relevant to genes or promotor regions for diagnostic or therapeutic purposes. When existing methylation assays incorrectly detect methylation-level values of cytosines in such genomic sites, the downstream diagnosis or therapeutic recommendations can likewise be inaccurate. In contrast to existing systems, in some embodiments, the bias-adjusted-methylation-assay system identifies a genomic coordinate or a genomic region of a sample comprising one or more target cytosine bases for which a given methylation assay has inaccurately measured methylation. For instance, the bias-adjusted-methylation-assay system can identify genomic coordinates for genes, portions of genes, promoter regions, or other genomic code that are subject to a bias in methylation detection by the given methylation assay.

In part due to such improved methylation-level values and improved identification of genomic coordinates with certain methylated cytosine bases, in certain implementations, the bias-adjusted-methylation-assay system improves the computing efficiency and processing time consumed by specialized sequencing devices and/or computing devices running analysis software that perform methylation assays. As noted above, some existing sequencing systems re-run methylation assays on multiple samples or run different types of methylation assays to detect cytosine methylation more reliably. Rather than perform redundant or time-intensive processing on specialized sequencing devices, the bias-adjusted-methylation-assay system can apply a methylation-bias-adjustment-machine-learning model to contextual sequences to correct for the bias of existing methylation assays-thereby obviating methylation-assay re-runs or diversified methylation-assay types. By introducing a first-of-its-kind machine-learning model, the bias-adjusted-methylation-assay system can adjust methylation-level values that reflect the chemical unpredictability, imaging inaccuracies, or other failures of existing methylation assays.

Beyond improved accuracy for methylation-level values or genomic-site identifications, in some embodiments, the bias-adjusted-methylation-assay system improves diagnosis of certain disorders or diseases. For instance, in some cases, the bias-adjusted-methylation-assay system can adjust methylation-level values for a particular genomic region from a given methylation assay to exceed or satisfy a hypomethylation threshold or a hypermethylation threshold indicative of a tumor type or other disorder. As a further example, in some cases, the bias-adjusted-methylation-assay system can adjust methylation-level values for a particular genomic region typically methylated by a particular enzyme, such as Methylenetetrahydrofolate reductase (MTHFR), and determine whether the adjusted methylation-level values indicate an activity level consistent with normal or abnormal activity of the particular enzyme. Based on determining whether the adjusted methylation-level values indicate an enzyme activity level, in some cases, the bias-adjusted-methylation-assay system can indicate whether certain therapeutic drugs or other treatment are suitable for the enzyme activity level.

As illustrated by the foregoing discussion, the present disclosure utilizes a variety of terms to describe features and advantages of the bias-adjusted-methylation-assay system. As used herein, for example, the term “methylation assay” refers to an assay that detects, measures, or quantifies methylation of cytosine from an oligonucleotide or other nucleotide sequence. In some cases, a methylation assay detects or quantifies methylation of cytosine at particular target genomic regions or in particular cell types. As suggested above and explained below, some methylation assays quantify methylation in terms of methylation-level values.

Relatedly, the term “methylation-level value” refers to a numeric value indicating an amount, percentage, ratio, or quantity of cytosine to which a methyl group or hydroxymethyl group has been added or bonded. For instance, a methylation-level value includes a score (e.g., ranging from 0 to 1) that indicates a percentage or ratio of cytosine bases (e.g., at CpG sites) for particular genomic coordinates or genomic regions to which a methyl group has been added. In some cases, a methylation-level value is expressed as a beta value or an M value. To illustrate, a beta value may estimate a methylation level using a ratio of signal intensities between methylated alleles corresponding to a genomic coordinate and unmethylated alleles corresponding to the genomic coordinate, where 0 represents completely unmethylated and 1 represents completely methylated. By contrast, an M value may represent a log2 ratio of signal intensities of a methylated probe and an unmethylated probe corresponding to a cytosine base.

As further used herein, the term “sample nucleotide sequence” refers to a sequence of nucleotides isolated or extracted from a sample organism (or a copy of such an isolated or extracted sequence). In particular, a sample nucleotide sequence includes a segment of a nucleic acid polymer that is isolated or extracted from a sample organism and composed of nitrogenous heterocyclic bases. For example, a sample nucleotide sequence can include a segment of deoxyribonucleic acid (DNA), ribonucleic acid (RNA), or other polymeric forms of nucleic acids or chimeric or hybrid forms of nucleic acids noted below. More specifically, in some cases, the sample nucleotide sequence is found in a sample prepared or isolated by a kit and received by a sequencing device.

As used herein, the term “machine-learning model” refers to a computer algorithm or a collection of computer algorithms that automatically improve performing a particular task through experience based on use of data. For example, a machine-learning model can utilize one or more learning techniques to improve in accuracy and/or effectiveness. Example machine-learning models include various types of decision trees, support vector machines, Bayesian networks, or neural networks. In some cases, the methylation-bias-adjustment-machine-learning model constitutes a deep neural network (e.g., convolutional neural network) or a series of decision trees (e.g., random forest, XGBoost), while in other cases the methylation-bias-adjustment-machine-learning model constitutes a multilayer perceptron, a linear regression, a support vector machine, a deep tabular learning architecture, a deep learning transformer (e.g., self-attention-based-tabular transformer), or a logistic regression.

In some cases, the bias-adjusted-methylation-assay system utilizes a methylation-bias-adjustment-machine-learning model to determine a bias score or a predicted methylation-level value for a contextual sequence. As used herein, the term “methylation-bias-adjustment-machine-learning model” refers to a machine-learning model that determines a value indicating an adjustment of a methylation-level value reflecting bias of a given methylation assay when measuring or quantifying methylation of cytosine. For example, in some cases, the methylation-bias-adjustment-machine-learning model is trained to generate a predicted methylation-level value of one or more cytosine bases when flanked or surrounded by a specific contextual sequence of nucleotides. As a further example, in some cases, the methylation-bias-adjustment-machine-learning model is trained to generate a bias score indicating a degree to which a methylation-level value from a given methylation assay errs or is incorrect for one or more cytosine bases flanked or surrounded by a specific contextual sequence of nucleotides.

Relatedly, the term “bias score” refers to a numerical value or classification indicating a degree to which a given methylation assay errs in detecting or quantifying methylation of one or more cytosine bases. For example, in some cases, a bias score includes a scaling factor indicating a degree to which a methylation-level value from a given methylation assay errs in detecting or quantifying one or more cytosine bases when flanked or surrounded by a contextual sequence. Accordingly, a bias score can be specific to a contextual sequence.

As further used herein, the term “contextual sequence” refers to a series of nucleobases from a sample nucleotide sequence (or other nucleotide sequence) that surround (e.g., flank on each side or neighbor) a target cytosine base or a target cytosine-guanine-dinucleotide pair at a CpG site. In some examples, a contextual sequence refers to a series of upstream and downstream nucleobases from a sample nucleotide sequence (e.g., a sample’s genome) that flank or surround a target cytosine base or a target cytosine-guanine-dinucleotide pair. Accordingly, a contextual sequence includes nucleobases from a sample nucleotide sequence that are located both upstream and downstream from a genomic coordinate(s) for a target cytosine base or a target cytosine-guanine-dinucleotide pair. As suggested throughout, a contextual sequence may include the five, ten, fifteen, or other threshold number of nucleobases upstream of a target cytosine base and the five, ten, or fifteen or other threshold number of nucleobases downstream from the target cytosine as determined from nucleotide-fragment reads from a sample. While the number of nucleobases upstream and downstream from a target cytosine base or from a target cytosine-guanine-dinucleotide pair may be equal to each other (e.g., 15 upstream nucleobases and 15 downstream nucleobases), in some embodiments, a contextual sequence includes numbers of nucleobases upstream and downstream from the target cytosine base or from the target cytosine-guanine-dinucleotide pair that do not equal each other (e.g., 14 upstream nucleobases and 15 downstream nucleobases).

As further used herein, the term “nucleotide-fragment read” (or simply “read”) refers to an inferred sequence of one or more nucleobases (or nucleobase pairs) from all or part of a sample nucleotide sequence (e.g., a sample genomic sequence, cDNA). In particular, a nucleotide-fragment read includes a determined or predicted sequence of nucleobase calls for a nucleotide sequence (or group of monoclonal nucleotide sequences) from a sample library fragment corresponding to a genome sample. For example, in some cases, a sequencing device determines a nucleotide-fragment read by generating nucleobase calls for nucleobases passed through a nanopore of a nucleotide-sample slide, determined via fluorescent tagging, or determined from a cluster in a flow cell.

As further used herein, the term “nucleobase call” (or simply “base call”) refers to a determination or prediction of a particular nucleobase (or nucleobase pair) for an oligonucleotide (e.g., read) during a sequencing cycle or for a genomic coordinate of a sample genome. In particular, a nucleobase call can indicate (i) a determination or prediction of the type of nucleobase that has been incorporated within an oligonucleotide on a nucleotide-sample slide (e.g., read-based nucleobase calls) or (ii) a determination or prediction of the type of nucleobase that is present at a genomic coordinate or region within a genome, including a variant call or a non-variant call in a digital output file. In some cases, for a nucleotide-fragment read, a nucleobase call includes a determination or a prediction of a nucleobase based on intensity values resulting from fluorescent-tagged nucleotides added to an oligonucleotide of a nucleotide-sample slide (e.g., in a cluster of a flow cell). Alternatively, a nucleobase call includes a determination or a prediction of a nucleobase from chromatogram peaks or electrical current changes resulting from nucleotides passing through a nanopore of a nucleotide-sample slide. By contrast, a nucleobase call can also include a final prediction of a nucleobase at a genomic coordinate of a sample genome for a variant call file (VCF) or other base-call-output file-based on nucleotide-fragment reads corresponding to the genomic coordinate. Accordingly, a nucleobase call can include a base call corresponding to a genomic coordinate and a reference genome, such as an indication of a variant or a non-variant at a particular location corresponding to the reference genome. Indeed, a nucleobase call can refer to a variant call, including but not limited to, a single nucleotide variant (SNV), an insertion or a deletion (indel), or base call that is part of a structural variant. As suggested above, a single nucleobase call can be an adenine (A) call, a cytosine (C) call, a guanine (G) call, or a thymine (T) call.

Relatedly, the term “nucleobase class” refers to a particular type or kind of nitrogenous base. For instance, a genome or nucleotide sequence may include five different nucleobase classes, including adenine (A), cytosine (C), guanine (G), or thymine (T), or uracil (U).

As further used herein, the term “genomic coordinate” (or sometimes simply “coordinate”) refers to a particular location or position of a nucleobase within a genome (e.g., an organism’s genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleobase within the source for the reference genome (e.g., mt: 16568 or SARS-CoV-2:29001). By contrast, in certain cases, a genomic coordinate refers to a position of a nucleobase within a reference genome without reference to a chromosome or source (e.g., 29727).

As mentioned above, a “genomic region” refers to a range of genomic coordinates. Like genomic coordinates, in certain embodiments, a genomic region may be identified by an identifier for a chromosome and a particular position or positions, such as numbered positions following the identifier for a chromosome (e.g., chr1:1234570-1234870).

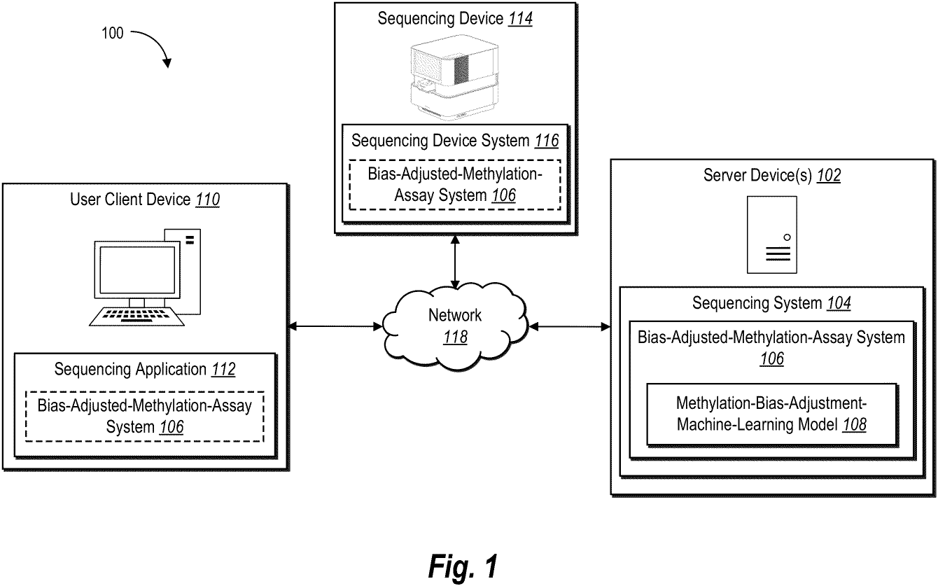

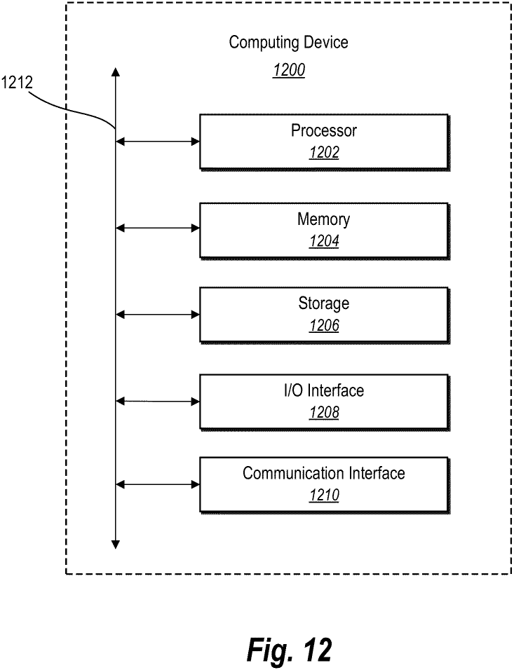

The following paragraphs describe the bias-adjusted-methylation-assay system with respect to illustrative figures that portray example embodiments and implementations. For example, FIG. 1 illustrates a schematic diagram of a computing system 100 in which a bias-adjusted-methylation-assay system 106 operates in accordance with one or more embodiments. As illustrated, the computing system includes server device(s) 102, a sequencing device 114, and a user client device 110 connected via a network 118. While FIG. 1 shows an embodiment of the bias-adjusted-methylation-assay system 106, this disclosure describes alternative embodiments and configurations below. As shown in FIG. 1, the sequencing device 114, the server device(s) 102, and the user client device 110 can communicate with each other via the network 118. The network 118 comprises any suitable network over which computing devices can communicate. Example networks are discussed in additional detail below with respect to FIG. 12.

As indicated by FIG. 1, the sequencing device 114 comprises a sequencing device system 116 for sequencing a genomic sample or other nucleic-acid polymer, such as when sequencing oligonucleotides extracted from the genomic sample as part of a methylation assay. In some embodiments, by executing the sequencing device system 116, the sequencing device 114 analyzes nucleic-acid segments or oligonucleotides extracted from genomic samples to generate nucleotide-fragment reads or other data utilizing computer implemented methods and systems (described herein) either directly or indirectly on the sequencing device 114. More particularly, the sequencing device 114 receives nucleotide-sample slides (e.g., flow cells) comprising nucleotide sequences extracted from samples and then copies and determines the nucleobase sequence of such extracted nucleotide sequences. As part of a methylation assay, for instance, the sequencing device 114 may determine nucleobase calls for nucleotide-fragment reads comprising CpG sites.

As suggested above, by executing the sequencing device system 116, the sequencing device 114 can run one or more sequencing cycles as part of a sequencing run. By executing the bias-adjusted-methylation-assay system 106, for instance, the sequencing device 114 can sequence uracil bases that were converted from methylated cytosine bases and that are part of a nucleotide-fragment read and determine nucleobase calls of thymine for such uracil bases as part of a methylation assay. In one or more embodiments, the sequencing device 114 utilizes Sequencing by Synthesis (SBS) to sequence nucleic-acid polymers into nucleotide-fragment reads.

In some cases, the server device(s) 102 is located at or near a same physical location of the sequencing device 114 or remotely from the sequencing device 114. Indeed, in some embodiments, the server device(s) 102 and the sequencing device 114 are integrated into a same computing device. The server device(s) 102 may run a sequencing system 104 or the bias-adjusted-methylation-assay system 106 to generate, receive, analyze, store, and transmit digital data, such as by receiving base-call data or determining variant calls based on analyzing such base-call data.

As suggested by FIG. 1, the sequencing device 114 may send (and the server device(s) 102 may receive) base-call data generated during a sequencing run of the sequencing device 114. By executing software in the form of the sequencing system 104 or the bias-adjusted-methylation-assay system 106, the server device(s) 102 may align nucleotide-fragment reads with a reference genome and determine variant calls based on the aligned nucleotide-fragment reads. The server device(s) 102 may also communicate with the user client device 110. In particular, the server device(s) 102 can send data to the user client device 110, including a variant call file (VCF), or other information indicating nucleobase calls, sequencing metrics, error data, or other metrics.

In some embodiments, the server device(s) 102 comprise a distributed collection of servers where the server device(s) 102 include a number of server devices distributed across the network 118 and located in the same or different physical locations. Further, the server device(s) 102 can comprise a content server, an application server, a communication server, a web-hosting server, or another type of server.

As further illustrated and indicated in FIG. 1, the user client device 110 can generate, store, receive, and send digital data. In particular, the user client device 110 can receive variant calls and corresponding sequencing metrics from the server device(s) 102 or receive base-call data (e.g., BCL or FASTQ) and corresponding sequencing metrics from the sequencing device 114. Furthermore, the user client device 110 may communicate with the server device(s) 102 or the server device(s) 102 to receive a VCF comprising nucleobase calls and/or other metrics, such as a base-call-quality metrics or pass-filter metrics. The user client device 110 can accordingly present or display information pertaining to variant calls or other nucleobase calls within a graphical user interface to a user associated with the user client device 110. In particular, the user client device 110 can present results from a methylation assay or graphics that indicate changes to bias scores specific to contextual sequences flanking target cytosine bases.

Although FIG. 1 depicts the user client device 110 as a desktop or laptop computer, the user client device 110 may comprise various types of client devices. For example, in some embodiments, the user client device 110 includes non-mobile devices, such as desktop computers or servers, or other types of client devices. In yet other embodiments, the user client device 110 includes mobile devices, such as laptops, tablets, mobile telephones, or smartphones. Additional details regarding the user client device 110 are discussed below with respect to FIG. 12.

As further illustrated in FIG. 1, the user client device 110 includes a sequencing application 112. The sequencing application 112 may be a web application or a native application stored and executed on the user client device 110 (e.g., a mobile application, desktop application). The sequencing application 112 can include instructions that (when executed) cause the user client device 110 to receive data from the bias-adjusted-methylation-assay system 106 and present, for display at the user client device 110, base-call data (e.g., from a BCL), data from a VCF, or data from a methylation assay.

As further illustrated in FIG. 1, a version of the bias-adjusted-methylation-assay system 106 may be located on the user client device 110 as part of the sequencing application 112 or on the sequencing device 114. Accordingly, in some embodiments, the bias-adjusted-methylation-assay system 106 is implemented by (e.g., located entirely or in part) on the user client device 110. In yet other embodiments, the bias-adjusted-methylation-assay system 106 is implemented by one or more other components of the computing system 100, such as the sequencing device 114. In particular, the bias-adjusted-methylation-assay system 106 can be implemented in a variety of different ways across the sequencing device 114, the user client device 110, and the server device(s) 102. For example, the bias-adjusted-methylation-assay system 106 can be downloaded from the server device(s) 102 to the sequencing device 114 and/or the user client device 110 where all or part of the functionality of the bias-adjusted-methylation-assay system 106 is performed at each respective device within the computing system.

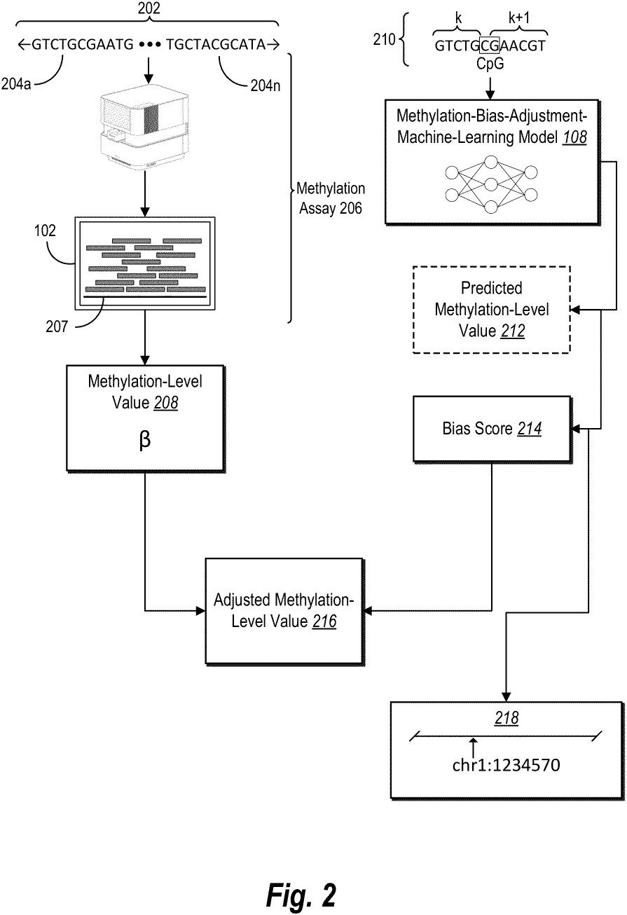

As indicated above, the bias-adj usted-methylation-assay system 106 can use a methylation-bias-adjustment-machine-learning model to determine factors indicating an error level with which a methylation assay detects methylation of cytosine bases when flanked by specific contextual sequences. In accordance with one or more embodiments, FIG. 2 illustrates an example of the bias-adjusted-methylation-assay system 106 (i) identifying a methylation-level value for a cytosine base within a sample nucleotide sequence determined by a given methylation assay, (ii) using a methylation-bias-adjustment-machine-learning model to generate a bias score indicating a degree to which the given methylation assay errs in detecting methylation of the cytosine base when flanked by a specific sequence context, and (iii) determining an adjusted methylation-level value for the cytosine base based on the bias score.

As shown in FIG. 2, for instance, the bias-adjusted-methylation-assay system 106 identifies a methylation-level value 208 for a cytosine base 204a within a sample nucleotide sequence 202 determined by a methylation assay 206 by either (i) accessing or receiving the methylation-level value 208 from a computing device or (ii) determining the methylation-level value 208 for the cytosine base 204a using the methylation assay 206. For example, in some cases, the bias-adjusted-methylation-assay system 106 inputs or runs the sample nucleotide sequence 202 through the methylation assay 206, such as TAPS. As indicated, the sample nucleotide sequence 202 comprises one or more cytosine bases at CpG sites, including the cytosine base 204a and a cytosine base 204n. In certain cases, the sample nucleotide sequence 202 constitutes a sample library fragment with genomic DNA from a sample comprising the cytosine base 204a and the cytosine base 204n. Consistent with the disclosure above, in certain implementations, the bias-adjusted-methylation-assay system 106 uses an enzyme to convert the cytosine bases 204a-204n to uracil bases as part of the methylation assay 206.

As further part of the methylation assay 206, in some embodiments, the bias-adjusted-methylation-assay system 106 amplifies and determines nucleobase calls for the sample nucleotide sequence 202 and complimentary strands using the sequencing device 114. In some such cases, the bias-adjusted-methylation-assay system 106 uses SBS to determine nucleobase calls for the sample nucleotide sequence 202 when sequencing or amplifying a nucleotide-fragment read, including thymine nucleobase calls for the cytosine bases 204a-204n that have been converted into uracil bases. Along with other determined nucleotide-fragment reads, in some cases, the sequencing device 114 sends base-call data to the server device(s) 102. As further indicated by FIG. 2, in certain implementations, the bias-adjusted-methylation-assay system 106 uses the server device(s) 102 to align the nucleotide-read fragments with a reference genome 207 and determine variant calls. As part of the methylation assay 206, for instance, the bias-adjusted-methylation-assay system 106 identifies thymine bases corresponding to the cytosine bases 204a-204n that vary from cytosine bases at CpG sites within the reference genome 207.

As further shown in FIG. 2, the bias-adjusted-methylation-assay system 106 determines the methylation-level value 208 for the cytosine base 204a as part of the methylation assay 206. For instance, in some cases, the bias-adjusted-methylation-assay system 106 determines a beta value indicating a percentage or ratio nucleotide-fragment reads covering the cytosine base 204a to which a methyl group or hydroxymethyl group has been added to the cytosine base 204a. In particular, the beta value may estimate a methylation level using a ratio of signal intensities between methylated alleles corresponding to a genomic coordinate for the cytosine base 204a and unmethylated alleles corresponding to the genomic coordinate for the cytosine base 204a. Alternatively, the methylation-level value 208 may constitute an M value that indicates a log2 ratio of signal intensities of a methylated probe corresponding to the cytosine base 204a and an unmethylated probe corresponding to the cytosine base 204a.

In addition to determining or otherwise identifying the methylation-level value 208 for the cytosine base 204a, as further shown in FIG. 2, the bias-adjusted-methylation-assay system 106 inputs data representing a contextual sequence 210 flanking the cytosine base 204a into the methylation-bias-adjustment-machine-learning model 108. In this particular example, the contextual sequence 210 includes five upstream nucleobases and six downstream nucleobases from a target cytosine base at a CpG site. Put differently, the contextual sequence 210 includes five upstream nucleobases and five downstream nucleobases from a target cytosine-guanine-dinucleotide pair at a CpG site.

To process the contextual sequence 210, the bias-adjusted-methylation-assay system 106 converts the contextual sequence 210 into a matrix, feature vector, or feature map representing the contextual sequence 210. Based on the data representing the contextual sequence 210, the methylation-bias-adjustment-machine-learning model 108 determines a value (e.g., predicted methylation-level value, bias score) indicating an adjustment of the methylation-level value 208 for a bias of the methylation assay 206 when measuring or quantifying methylation of cytosine. As suggested by FIG. 2, the bias-adjusted-methylation-assay system 106 can determine such a value by either directly or indirectly determining a bias score 214 indicating a degree to which the methylation-level value 208 errs in detecting or measuring methylation of the cytosine base 204a.

In some embodiments, for instance, the methylation-bias-adjustment-machine-learning model 108 generates a predicted methylation-level value 212. When trained as a CNN or other neural network, for example, the methylation-bias-adjustment-machine-learning model 108 generates the predicted methylation-level value 212 representing a level of methylation of the cytosine base 204a when surrounded by the contextual sequence 210. To determine the bias score 214, in some such cases, the bias-adjusted-methylation-assay system 106 determines a value difference between the predicted methylation-level value 212 and an expected methylation-level value (not shown) for the cytosine base 204a flanked by the contextual sequence 210 within a synthetically methylated nucleotide sequence. Alternatively, to determine the bias score 214, the bias-adjusted-methylation-assay system 106 determines a value difference between the predicted methylation-level value 212 and the methylation-level value 208.

By contrast, in some embodiments, the methylation-bias-adjustment-machine-learning model 108 generates the bias score 214. When trained as a series or decision trees (e.g., random forest), for instance, the methylation-bias-adjustment-machine-learning model 108 generates the bias score 214 based on the input data representing the contextual sequence 210. Regardless of whether the bias score 214 is determined directly or indirectly by the methylation-bias-adjustment-machine-learning model 108, the bias score 214 can be specific to the nucleobases in the contextual sequence 210. In other words, the bias score 214 indicates a degree to which a specific sequence of upstream and downstream nucleobases affects or skew the methylation assay 206 in measuring or quantifying the level of methylation of the cytosine base 204a.

Based on the bias score 214, as further shown in FIG. 2, the bias-adjusted-methylation-assay system 106 adjusts the methylation-level value 208 to generate an adjusted methylation-level value 216. For instance, in some cases, the bias-adjusted-methylation-assay system 106 multiplies the methylation-level value 208 by the bias score 214 (which can represent a scaling factor) to determine the adjusted methylation-level value 216. In other embodiments, however, the bias-adjusted-methylation-assay system 106 can use the bias score 214 to adjust the methylation-level value 208 in other suitable ways, including, but not limited to, dividing the methylation-level value 208 by the bias score 214 or adding the bias score 214 to (or subtracting the bias score 214 from) the methylation-level value 208.

In addition to determining the bias score 214, in some embodiments, the bias-adjusted-methylation-assay system 106 determines or identifies a genomic coordinate 218 or genomic region for the cytosine base 204a subject to a bias indicated by the bias score 214. For example, in some cases, the bias-adjusted-methylation-assay system 106 identifies (and provides for display within a graphical user interface) the genomic coordinate 218 for the cytosine base 204a flanked by nucleobases following or mirroring the contextual sequence 210. The bias-adjusted-methylation-assay system 106 may accordingly identify multiple genomic coordinates for cytosine bases flanked by upstream and downstream nucleobases that follow or mirror the contextual sequence 210.

While FIG. 2 depicts the bias-adjusted-methylation-assay system 106 determining and adjusting a methylation-level value for the cytosine base 204a flanked by a specific contextual sequence, as indicated above, the bias-adjusted-methylation-assay system 106 can likewise determine and adjust methylation-level values for multiple cytosine bases respectively flanked by different contextual sequences, such as the cytosine base 204n. Indeed, in some cases, the bias-adjusted-methylation-assay system 106 uses the methylation assay 206 to sequentially or concurrently determine methylation-level values indicating methylation levels of additional cytosine bases within the same or different sample nucleotide sequences. The bias-adjusted-methylation-assay system 106 further uses the methylation-bias-adjustment-machine-learning model 108 to determine bias scores for contextual sequences respectively flanking the cytosine bases, such as the cytosine base 204n. Based on the bias scores, the disclosed system can adjust the methylation-level values output by the methylation assay 206 and/or identify genomic coordinates or regions for the cytosine bases subject to a bias of the methylation assay 206.

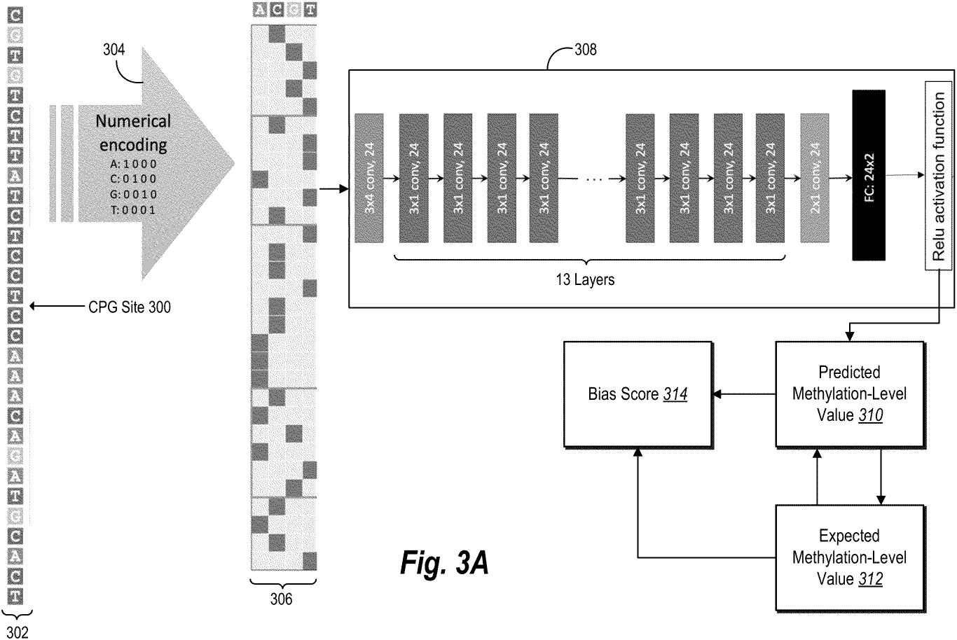

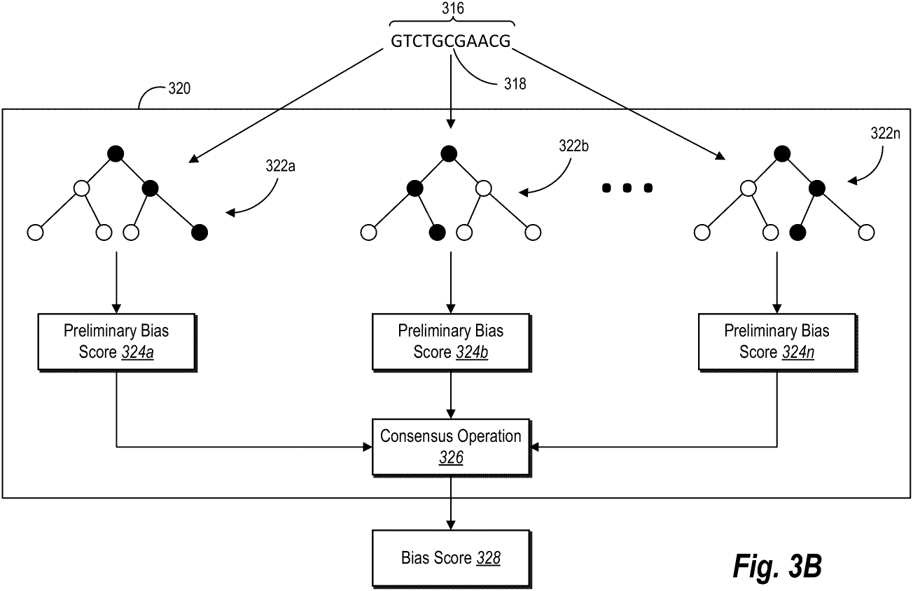

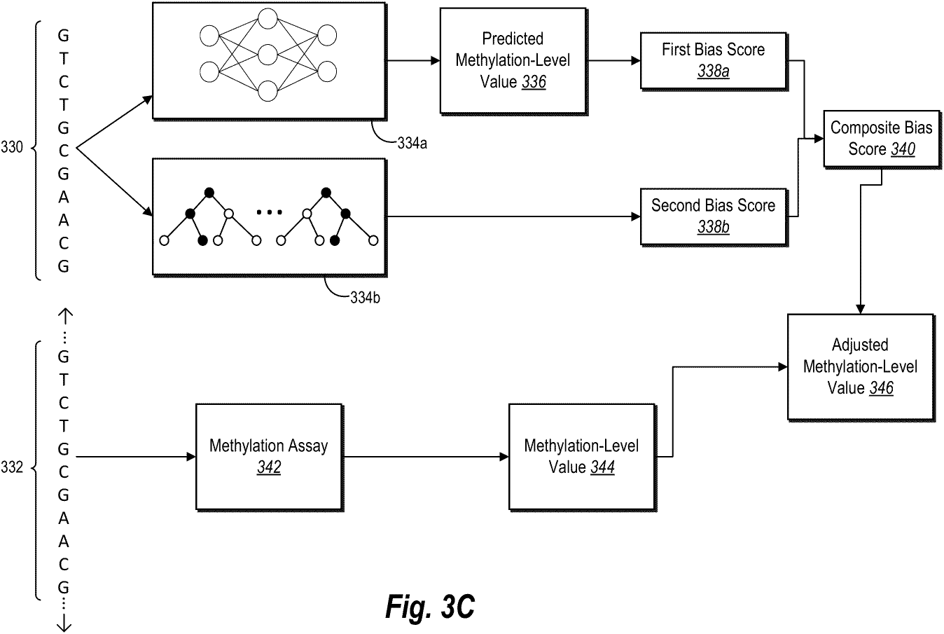

As suggested above, a methylation-bias-adjustment-machine-learning model can take a variety of forms and include multiple machine-learning models. In accordance with one or more embodiments, FIGS. 3A-3C illustrate different implementations of a methylation-bias-adjustment-machine-learning model. For instance, FIG. 3A depicts the bias-adjusted-methylation-assay system 106 utilizing a neural network as the methylation-bias-adjustment-machine-learning model to generate a predicted methylation-level value and subsequently determining a bias score for a specific contextual sequence. FIG. 3B depicts the bias-adjusted-methylation-assay system 106 utilizing a random-forest model as the methylation-bias-adjustment-machine-learning model to generate a bias score for a specific contextual sequence. FIG. 3C depicts the bias-adjusted-methylation-assay system 106 utilizing a combination of different methylation-bias-adjustment-machine-learning models together to generate a composite bias score for a specific contextual sequence.

As just indicated, FIG. 3A depicts the bias-adjusted-methylation-assay system 106 utilizing a CNN as a methylation-bias-adjustment-machine-learning model 308. As shown in FIG. 3A, for instance, the bias-adjusted-methylation-assay system 106 inputs a dataset 306 representing a contextual sequence 302 flanking a cytosine base of a CpG site 300 into the methylation-bias-adjustment-machine-learning model 308, the methylation-bias-adjustment-machine-learning model 308 generates a predicted methylation-level value 310 for the contextual sequence 302, and the bias-adjusted-methylation-assay system 106 determines a bias score 314 based on a comparison of the predicted methylation-level value 310 and an expected methylation-level value 312 for the cytosine base when flanked by the contextual sequence 302 within a synthetically methylated nucleotide sequence.

As just indicated and as depicted in FIG. 3A, the bias-adjusted-methylation-assay system 106 processes the contextual sequence 302 comprising the cytosine base at the CpG site 300. In this particular example, FIG. 3A does not depict the cytosine and guanine bases for the CpG site 300 flanked by the contextual sequence 302. As illustrated without the CpG site 300 shown, the contextual sequence 302 comprises fifteen nucleobases upstream from the CpG site 300 and fifteen nucleobases downstream from the CpG site 300. But other numbers of upstream and downstream nucleobases may be used for a contextual sequence, as explained further below.

To process the contextual sequence 302, in some embodiments, the bias-adjusted-methylation-assay system 106 performs an encoding algorithm 304 to transform the contextual sequence 302 from nucleobases (or letters representing nucleobases) into a dataset 306 representing the contextual sequence 302. For instance, the bias-adjusted-methylation-assay system 106 can perform one-hot coding as the encoding algorithm 304 to transform the letters to a feature map as the dataset 306. The bias-adjusted-methylation-assay system 106 further inputs the dataset 306 representing the contextual sequence 302 into the methylation-bias-adjustment-machine-learning model 308.

As depicted in FIG. 3A, a CNN with a customized architecture constitutes the methylation-bias-adjustment-machine-learning model 308. In particular, the customized CNN comprises approximately fifteen convolutional layers each with twenty-four channels and a convolutional kernel size of three, followed by a fully connected layer and a rectified linear unit (ReLu) layer. While the methylation-bias-adjustment-machine-learning model 308 depicted in FIG. 3A includes a certain number of convolutional layers for a contextual sequence with fifteen upstream and fifteen downstream nucleobases flanking a CpG site, the bias-adjusted-methylation-assay system 106 may adjust the number of convolutional layers and other layer parameters of a CNN for a different length of contextual sequence. In some cases, the bias-adjusted-methylation-assay system 106 uses batch normalization and ReLu activations respectively before and after each convolutional layer. As suggested above, a different CNN or different neural network may likewise be used as the methylation-bias-adjustment-machine-learning model 308 with different layers.

In some cases, a CNN, such as that depicted in FIG. 3A, outperforms other deep neural networks because the input patterns for a contextual sequence are local and the input datasets are relatively smaller in size-without a need to capture long range dependencies. As shown in the customized CNN of FIG. 3A, the methylation-bias-adjustment-machine-learning model 308 lacks pooling layers to ensure that the CNN includes layers that detect and recognize location sensitivity for a contextual sequence of different nucleobase classes (e.g., A, T, C, G). To compensate for a lack of pooling layers and increase receptivity through the CNN, the methylation-bias-adjustment-machine-learning model 308 depicted in FIG. 3A includes un-padded convolutional kernels that gradually reduce a length of the feature map as the dataset 306. As further depicted in FIG. 3A, the customized CNN comprises convolutional layers with twenty-four channels that have been selected through experimentation to avoid overfitting the trained CNN to a training dataset and avoid computational complexity that might come with more channels (e.g., sixty-four channels)-while maintaining accurate predicted methylation-level values.

As trained, the methylation-bias-adjustment-machine-learning model 308 generates the predicted methylation-level value 310 indicating a level of methylation of the cytosine base from the CpG site 300-when flanked by the nucleobases within the contextual sequence 302. In this embodiment, therefore, the methylation-bias-adjustment-machine-learning model 308 outputs a type of adjusted methylation-level value that a given methylation assay should produce for the cytosine base at the CpG site 300-when processing sample nucleotide sequences comprising the cytosine base flanked by the contextual sequence 302.

As suggested above, the bias-adjusted-methylation-assay system 106 uses the predicted methylation-level value 310 to determine the bias score 314. To facilitate determining the bias score 314, in some embodiments, the bias-adjusted-methylation-assay system 106 determines the expected methylation-level value 312. In some cases, the expected methylation-level value 312 represents a ground truth methylation-level value for a synthetically methylated cytosine base flanked by the contextual sequence 302. Because an accurate methylation-level value for a synthetically methylated cytosine base would indicate or reflect fully methylated cytosine bases (e.g., from different alleles) at a particular genomic coordinate, such as the CpG site 300, the expected methylation-level value 312 can represent 100% methylation (e.g., a value of 1).

As further indicated by FIG. 3A, the bias-adjusted-methylation-assay system 106 compares the predicted methylation-level value 310 to the expected methylation-level value 312 for a cytosine base flanked by the contextual sequence 302 within a synthetically methylated nucleotide sequence. Alternatively, the bias-adjusted-methylation-assay system 106 compares the predicted methylation-level value 310 to a methylation-level value generated by a given methylation assay (not shown) for the cytosine base flanked by the contextual sequence 302 within a sample nucleotide sequence. Based on the comparison of the predicted methylation-level value 310 to the expected methylation-level value 312 or to a methylation-level value generated by the given methylation assay, the bias-adjusted-methylation-assay system 106 determines the bias score 314. As depicted here, the bias score 314 constitutes a value difference representing a predicted bias by which the given methylation assay errs in determining a methylation level of a cytosine base when flanked by the contextual sequence 302 in a sample nucleotide sequence.

While FIG. 3A depicts a single bias score, the methylation-bias-adjustment-machine-learning model 308 has been trained to generate bias scores for different contextual sequences. Accordingly, the methylation-bias-adjustment-machine-learning model 308 can generate predicted methylation-level values-and the bias-adjusted-methylation-assay system 106 determine corresponding bias scores-specific to different contextual sequences input as encoded datasets. Further, the bias-adjusted-methylation-assay system 106 can use bias scores generated by the methylation-bias-adjustment-machine-learning model 308 to adjust corresponding methylation-level values generated by the given methylation assay for cytosine bases flanked by corresponding contextual sequences.

As indicated above, FIG. 3B depicts the bias-adjusted-methylation-assay system 106 utilizing a random-forest model performing a regression as a methylation-bias-adjustment-machine-learning model 320. As shown in FIG. 3B, for instance, the bias-adjusted-methylation-assay system 106 inputs a dataset representing a contextual sequence 316 flanking a cytosine base 318 into the methylation-bias-adjustment-machine-learning model 320, and the methylation-bias-adjustment-machine-learning model 320 generates a bias score 328 for the contextual sequence 316 based on preliminary bias scores 324a, 324b, and 324n generated by decision trees 322a, 322b, and 322n.

As further shown in FIG. 3B, the bias-adjusted-methylation-assay system 106 processes the contextual sequence 316 comprising the cytosine base 318 at a CpG site and five nucleobases both upstream and downstream from the cytosine base 318. To process the contextual sequence 316, in some embodiments, the bias-adjusted-methylation-assay system 106 encodes the contextual sequence 316 from nucleobases (or letters representing nucleobases) into a dataset (e.g., feature vector, feature map).

When using a series of decision trees as a regressor, such as the methylation-bias-adjustment-machine-learning model 320, the bias-adjusted-methylation-assay system 106 can employ the series of decision trees to execute a regression. As indicated by FIG. 3B, in some embodiments, the methylation-bias-adjustment-machine-learning model 320 includes decision trees 322a-322n that each include different decision nodes and determine the preliminary bias scores 324a-324n, respectively. As explained below, various decision nodes within the decision trees 322a-322n have been trained to correctly determine in the aggregate a bias score indicating a degree to which a given methylation assay errs in detecting or quantifying methylation of cytosine bases when flanked by specific contextual sequences.

After the decision trees 322a-322n generate the preliminary bias scores 324a-324n, in certain implementations, the methylation-bias-adjustment-machine-learning model 320 performs a consensus operation 326 on the preliminary bias scores 324a-324n to generate the bias score 328 for the contextual sequence 316. For instance, in certain embodiments, the methylation-bias-adjustment-machine-learning model 320 averages (or determines a weighted average of) the preliminary bias scores 324a-324n to generate the bias score 328. By averaging or otherwise combining the preliminary bias scores 324a-324n, the bias score 328 represents a more accurate value that avoids overfitting to training data by any individual decision tree among the decision trees 322a-322n.

While FIG. 3B depicts a single bias score for a single contextual sequence, the methylation-bias-adjustment-machine-learning model 320 has been trained to generate bias scores for different contextual sequences. Accordingly, the methylation-bias-adjustment-machine-learning model 320 can generate different bias scores specific to different contextual sequences input as encoded datasets. Further, the bias-adjusted-methylation-assay system 106 can use bias scores generated by the methylation-bias-adjustment-machine-learning model 320 to adjust corresponding methylation-level values generated by a given methylation assay for cytosine bases flanked by corresponding contextual sequences.

As indicated above, in some embodiments, the bias-adjusted-methylation-assay system 106 combines or uses bias scores from multiple machine-learning models to generate a composite bias score. As depicted in FIG. 3C, in some embodiments, the bias-adjusted-methylation-assay system 106 uses a first methylation-bias-adjustment-machine-learning model 334a to determine a first bias score 338a for a contextual sequence 330 flanking a target cytosine base and a second methylation-bias-adjustment-machine-learning model 334b to determine a second bias score 338b for the contextual sequence 330. The bias-adjusted-methylation-assay system 106 can subsequently determine a composite bias score 340 for the contextual sequence 330-based on the first bias score 338a and the second bias score 338b-and subsequently adjust a methylation-level value 344 for the target cytosine base based on the composite bias score 340.

As shown in FIG. 3C, for instance, the bias-adjusted-methylation-assay system 106 passes a dataset representing the contextual sequence 330 through the first methylation-bias-adjustment-machine-learning model 334a. In some cases, the first methylation-bias-adjustment-machine-learning model 334a constitutes a neural network, such as a CNN. Based on the dataset representing the contextual sequence 330, the first methylation-bias-adjustment-machine-learning model 334a generates a predicted methylation-level value 336 for a cytosine base flanked by the contextual sequence 330. Consistent with the disclosure above, the bias-adjusted-methylation-assay system 106 further processes a nucleotide sequence 332 comprising the contextual sequence 330 through a methylation assay 342 to determine the methylation-level value 344. The bias-adjusted-methylation-assay system 106 subsequently determines, as the first bias score 338a for the contextual sequence 330, a value difference between the predicted methylation-level value 336 from the first methylation-bias-adjustment-machine-learning model 334a and an expected methylation-level value for a cytosine base flanked by the contextual sequence 330 within a synthetically methylated nucleotide sequence. Alternatively, the bias-adjusted-methylation-assay system 106 subsequently determines, as the first bias score 338a for the contextual sequence 330, a value difference between the predicted methylation-level value 336 from the first methylation-bias-adjustment-machine-learning model 334a and the methylation-level value 344 from the methylation assay 342.

As further shown in FIG. 3C, the bias-adjusted-methylation-assay system 106 passes a dataset representing the contextual sequence 330 through the second methylation-bias-adjustment-machine-learning model 334b. In some cases, the second methylation-bias-adjustment-machine-learning model 334b constitutes a decision-tree model, such as a random-forest model. Based on the dataset representing the contextual sequence 330, the second methylation-bias-adjustment-machine-learning model 334b generates the second bias score 338b for the contextual sequence 330. The bias-adjusted-methylation-assay system 106 subsequently combines the first bias score 338a and the second bias score 338b to generate the composite bias score 340 for the contextual sequence 330. For instance, the bias-adj usted-methylation-assay system 106 can determine an average, a weighted average, or other suitable combination of the first bias score 338a and the second bias score 338b to generate the composite bias score 340.

Finally, as further show in FIG. 3C, the bias-adjusted-methylation-assay system 106 adjusts the methylation-level value 344 based on the composite bias score 340 to generate an adjusted methylation-level value 346. For instance, in some embodiments, the bias-adjusted-methylation-assay system 106 multiplies or divides the methylation-level value 344 by the composite bias score 340. Depending on the form of the composite bias score 340, the bias-adjusted-methylation-assay system 106 may alternatively perform other operations using the composite bias score 340 with the methylation-level value 344 to generate the adjusted methylation-level value 346.

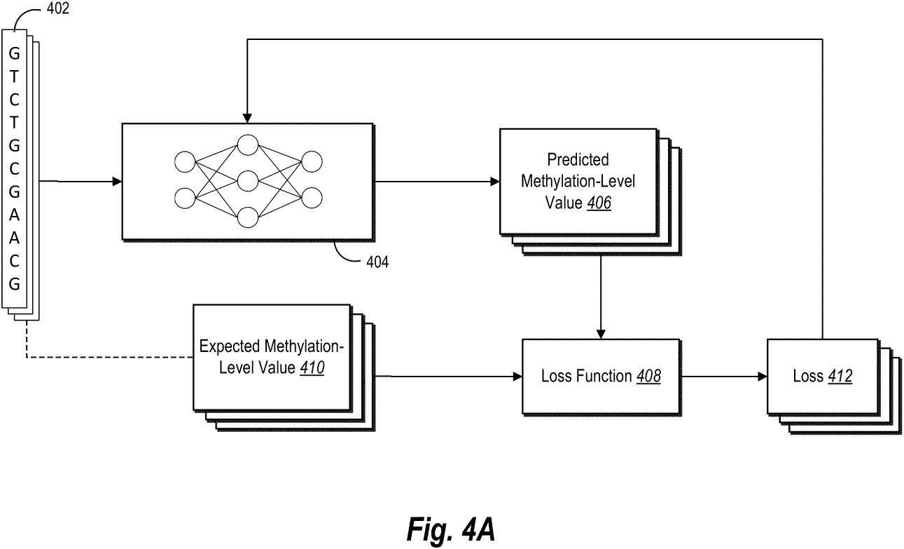

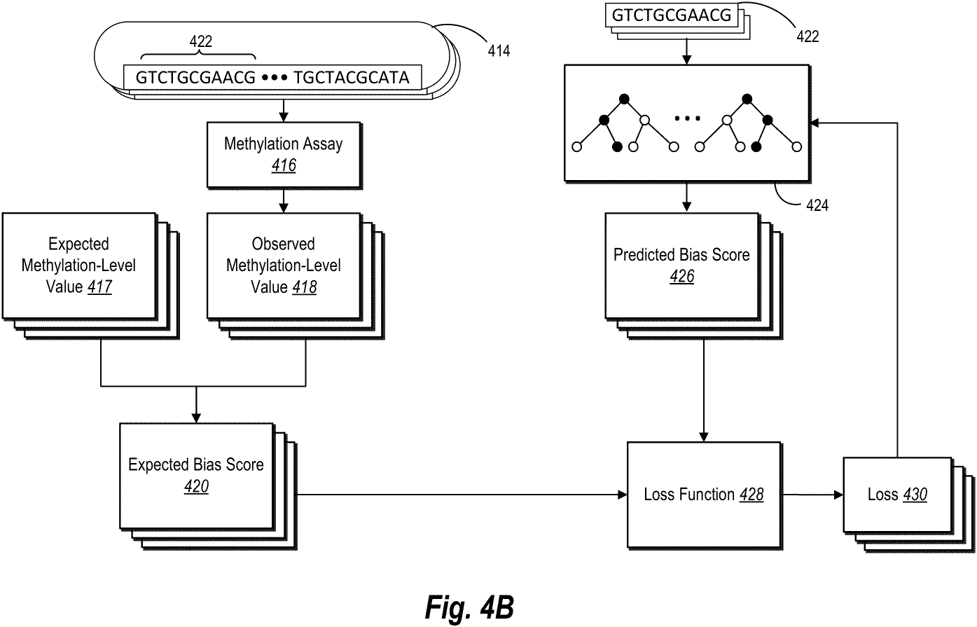

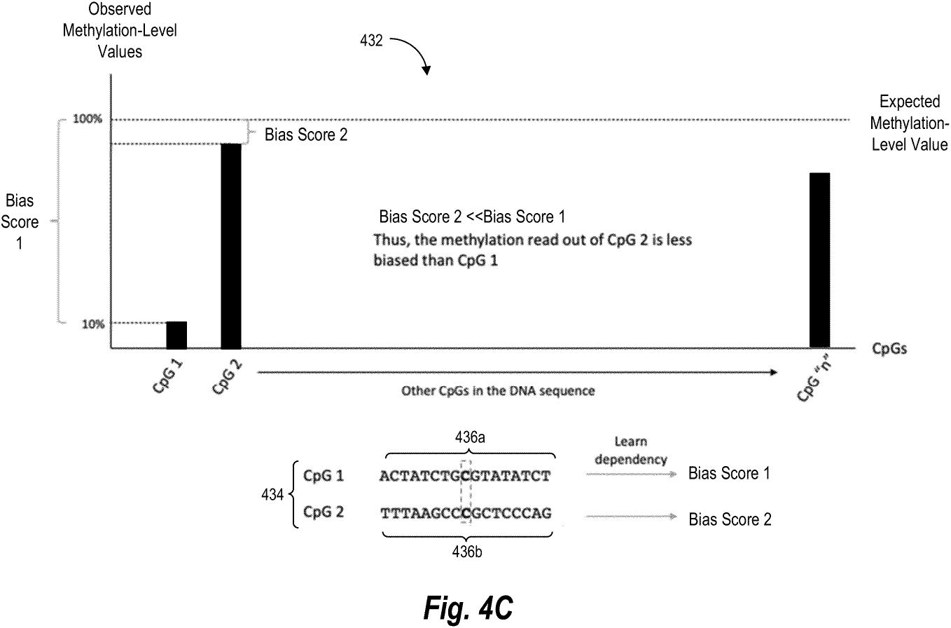

As noted above, in some embodiments, the bias-adjusted-methylation-assay system 106 trains a methylation-bias-adjustment-machine-learning model to determine values that are specific to contextual sequences and that indicate bias of a given methylation assay. In accordance with one or more embodiments, FIGS. 4A-4C depict the bias-adjusted-methylation-assay system 106 training different embodiments or different diagrams of a methylation-bias-adjustment-machine-learning model. For instance, FIG. 4A depicts the bias-adjusted-methylation-assay system 106 training a methylation-bias-adjustment-machine-learning model 404 as a neural network to determine methylation-level values that reflect a bias of a given methylation assay according to specific contextual sequences. FIG. 4B depicts the bias-adjusted-methylation-assay system 106 training a methylation-bias-adjustment-machine-learning model 424 as a decision-tree model to determine bias scores of a given methylation assay according to specific contextual sequences. FIG. 4C depicts the bias-adjusted-methylation-assay system 106 training a methylation-bias-adjustment-machine-learning model to learn bias scores reflecting a dependency between specific contextual sequences and error rate of a given methylation assay.

For simplicity, this disclosure describes an initial training iteration of a neural network followed by a summary of subsequent training iterations depicted in FIG. 4A. In an initial training iteration depicted by FIG. 4A, for example, the bias-adjusted-methylation-assay system 106 inputs into the methylation-bias-adjustment-machine-learning model 404 a training dataset representing a contextual sequence 402 flanking a cytosine base (e.g., training feature vector, training feature map). As indicated above, the contextual sequence 402 represents a specific sequence of upstream and downstream nucleobases from a target cytosine base, such as a cytosine base located at a CpG site. In some embodiments, the methylation-bias-adjustment-machine-learning model 404 constitutes a neural network with layers designed to process contextual sequences, such as the CNN depicted in FIG. 3A. As depicted in FIG. 4A and consistent with the disclosure above concerning neural networks, the methylation-bias-adjustment-machine-learning model 404 generates a predicted methylation-level value 406 based on the training dataset representing the contextual sequence 402. While this disclosure refers to the methylation-bias-adjustment-machine-learning model 404 as a neural network when generating a predicted methylation-level value, in some embodiments, the methylation-bias-adjustment-machine-learning model 404 constitutes a different type of machine-learning model, such as a decision-tree model.

As further shown in FIG. 4A, after determining the predicted methylation-level value 406, the bias-adjusted-methylation-assay system 106 compares the predicted methylation-level value 406 to an expected methylation-level value 410 for the contextual sequence 402. In some cases, the expected methylation-level value 410 represents a ground truth methylation-level value for a synthetically methylated cytosine base flanked by the contextual sequence 402. Because an accurate methylation-level value for a synthetically methylated cytosine base would indicate or reflect fully methylated cytosine bases (e.g., from different alleles) at a particular genomic coordinate, in some cases, the expected methylation-level value 410 represents 100% methylation (e.g., a value of 1).

In some implementations, the bias-adjusted-methylation-assay system 106 uses a loss function 408 to compare (and determine any difference) between the predicted methylation-level value 406 and the expected methylation-level value 410. As shown in FIG. 4A, the bias-adjusted-methylation-assay system 106 determines a loss 412 from the predicted methylation-level value 406 and the expected methylation-level value 410 utilizing the loss function 408. Because the loss 412 can be a difference between the predicted methylation-level value 406 and the expected methylation-level value 410, the loss 412 can represent a training value difference.

Depending on the form of the methylation-bias-adjustment-machine-learning model 404, the bias-adjusted-methylation-assay system 106 can use a variety of loss functions for the loss function 408. In certain embodiments, for instance, the bias-adjusted-methylation-assay system 106 uses a mean square error for a CNN, where a loss is determined using a value of 1 representing approximately 100% methylation of cytosine bases flanked by the contextual sequence 402. In particular, the bias-adjusted-methylation-assay system 106 uses the following function to determine the loss 412:

$$M S E = \frac{1}{n} {\sum }_{i = 1}^{n} {\left({Y}_{i} - {\hat{Y}}_{i}\right)}^{2} ,$$

where Yᵢ represents an expected (or ground-truth) methylation-level value for a contextual sequence, Ŷᵢ represents a predicted methylation-level value for the contextual sequence, and n represents a number of predictions (e.g., number of predicted methylation-level values). In contrast to using mean square error function, in some embodiments, the bias-adjusted-methylation-assay system 106 uses a logistic loss function (e.g., for a logistic regression model) or a cross-entropy-loss function or a least-squared-error function (e.g., for a LSTM).

Based on the determined loss 412 from the loss function 408, the bias-adjusted-methylation-assay system 106 adjusts parameters of the methylation-bias-adjustment-machine-learning model 404. By adjusting the parameters over training iterations, the bias-adjusted-methylation-assay system 106 increases the accuracy with which the methylation-bias-adjustment-machine-learning model 404 determines predicted methylation-level values. Based on the determined loss 412, for instance, the bias-adjusted-methylation-assay system 106 determines a gradient for weights using stochastic gradient descent (SGD). In some cases, the bias-adjusted-methylation-assay system 106 uses the following function:

$$w : = w - η ∇ Q \left(w\right) = w - \frac{η}{n} {\sum }_{i = 1}^{n} ∇ {Q}_{i} \left(w\right) ,$$

where w represents a weight of the methylation-bias-adjustment-machine-learning model 404 and ∇Qᵢ represents a gradient. After determining the gradient, the bias-adjusted-methylation-assay system 106 adjusts weights of the methylation-bias-adjustment-machine-learning model 404 based on the gradient in a given training iteration. In the alternative to SGD, the bias-adjusted-methylation-assay system 106 can use gradient descent or a different optimization method for training across training iterations.

After the initial training iteration and parameter adjustment, as indicated by FIG. 4A, the bias-adjusted-methylation-assay system 106 further determines predicted methylation-value levels for training datasets representing different contextual sequences. In some cases, the bias-adjusted-methylation-assay system 106 performs training iterations until the parameters (e.g., value or weights) of the methylation-bias-adjustment-machine-learning model 404 do not change significantly across training iterations or otherwise satisfy a convergence criteria.

Turning now to FIG. 4B, again for simplicity, this disclosure describes an initial training iteration of a decision-tree model followed by a summary of subsequent training iterations depicted in FIG. 4B. In an initial training iteration depicted by FIG. 4B, for example, the bias-adjusted-methylation-assay system 106 passes a nucleotide sequence 414 comprising one or more synthetically methylated cytosine bases through a methylation assay 416. To facilitate training the methylation-bias-adjustment-machine-learning model 424, the nucleotide sequence 414 includes a contextual sequence 422 flanking a cytosine base.

To synthetically methylate the nucleotide sequence 414, in some embodiments, the bias-adjusted-methylation-assay system 106 synthetically methylates each cytosine base (e.g., 100% CpGs) within oligonucleotides that each span approximately 2,500 nucleobases in length and are used for training. In some cases, for instance, the bias-adjusted-methylation-assay system 106 synthetically methylates cytosine bases within plasmids (e.g., pUC19) that are bioinformatically extracted from a larger DNA molecule. Indeed, as but one example, FIG. 4B depicts the nucleotide sequence 414 as part of a circular plasmid.

For training purposes, in some implementations, the oligonucleotides include various contextual sequences flanking cytosine bases. In one such embodiment, the bias-adjusted-methylation-assay system 106 uses a group of oligonucleotides for training that collectively include approximately 350 different contextual sequences flanking cytosine bases that have been synthetically methylated. As depicted in FIG. 4B, the nucleotide sequence 414 may represent one such synthetically methylated oligonucleotide.

As further shown in FIG. 4B, the bias-adjusted-methylation-assay system 106 uses a methylation assay 416 to determine an observed methylation-level value 418 for one or more cytosine bases within the nucleotide sequence 414. Because the methylation assay 416 can err in detecting or quantifying methylation of cytosine bases, in some cases, the methylation assay 416 generates the observed methylation-level value 418 with a degree of error exhibited by the methylation assay 416. During training iterations, the bias-adjusted-methylation-assay system 106 learns the bias of the methylation assay 416 according to specific contextual sequences flanking target cytosines.

As part of such training, the bias-adjusted-methylation-assay system 106 compares the observed methylation-level value 418 and an expected methylation-level value 417 for the synthetically methylated cytosine bases within the nucleotide sequence 414 to determine an expected bias score 420 of the methylation assay 416 for a specific contextual sequence. Because the nucleotide sequence 414 is synthetically methylated, the bias-adjusted-methylation-assay system 106 can a priori determine the expected methylation-level value 417. In some embodiments, the expected methylation-level value 417 indicates complete (or approximately complete) methylation of cytosine bases within the nucleotide sequence 414 (e.g., a beta value of 1). Accordingly, the bias-adjusted-methylation-assay system 106 determines the expected bias score 420 for the specific contextual sequence based on a comparison between the observed methylation-level value 418 and the expected methylation-level value 417. In some cases, the expected bias score 420 represents a factor by which the observed methylation-level value 418 is multiplied to produce the expected methylation-level value 417. By determining the expected bias score 420 for the specific contextual sequence, in some cases, the bias-adjusted-methylation-assay system 106 identifies a ground truth upon which it can train the methylation-bias-adjustment-machine-learning model 424 for a given training iteration.

As further shown in FIG. 4B, the bias-adjusted-methylation-assay system 106 inputs a training dataset representing the contextual sequence 422 into the methylation-bias-adjustment-machine-learning model 424. As indicated above, in some embodiments, the methylation-bias-adjustment-machine-learning model 424 represents a decision-tree model, such as a random-forest model. Consistent with the disclosure above concerning decision-tree models, the bias-adjusted-methylation-assay system 106 uses the methylation-bias-adjustment-machine-learning model 424 to generate a predicted bias score 426 for the contextual sequence 422. The predicted bias score 426 represents a predicted degree to which the methylation assay 416 errs in detecting or quantifying methylation of a cytosine base when flanked by the contextual sequence 422. While this disclosure refers to the methylation-bias-adjustment-machine-learning model 424 as a decision-tree model when generating a bias score, in some embodiments, the methylation-bias-adjustment-machine-learning model 424 constitutes a different type of machine-learning model, such as a neural network.

As further shown in FIG. 4B, the bias-adjusted-methylation-assay system 106 can compare the predicted bias score 426 and the expected bias score 420 as a basis for training the methylation-bias-adjustment-machine-learning model 424 to generate more accurate predicted bias scores. For instance, the bias-adjusted-methylation-assay system 106 compares (and determines any difference between) the predicted bias score 426 and the expected bias score 420 using a loss function 428. The difference between the predicted bias score 426 and the expected bias score 420 constitutes a loss 430. For example, in some embodiments, the bias-adjusted-methylation-assay system 106 determines a Gini importance metric or a Gini impurity metric as the loss 430 based on a comparison between the predicted bias score 426 and the expected bias score 420 using a Gini index as the loss function 428. In the alternative, in some embodiments, the bias-adjusted-methylation-assay system 106 determines a cross-entropy metric as the loss 430 based on a comparison between the predicted bias score 426 and the expected bias score 420-using a cross-entropy-loss function as the loss function 428.

Based on the determined loss 430 from the loss function 428, the bias-adjusted-methylation-assay system 106 subsequently adjusts parameters of the methylation-bias-adjustment-machine-learning model 424. By adjusting the parameters over training iterations, the bias-adjusted-methylation-assay system 106 increases the accuracy with which the methylation-bias-adjustment-machine-learning model 424 determines bias scores. To adjust parameters based on the determined loss 430, for instance, the bias-adjusted-methylation-assay system 106 adjusts one or more decision nodes of one or more decision trees within the methylation-bias-adjustment-machine-learning model 424. Additionally, or alternatively, the bias-adjusted-methylation-assay system 106 adds or removes one or more decision trees within the methylation-bias-adjustment-machine-learning model 424 as part of adjusting parameters.

After the initial training iteration and parameter adjustment, as shown by FIG. 4B, the bias-adjusted-methylation-assay system 106 continues to (i) use the methylation assay 416 to determine observed methylation-level values and expected methylation-level values for synthetically methylated nucleotide sequences comprising different contextual sequences flanking cytosine bases and determine corresponding expected bias scores for the different contextual sequences. Likewise, the bias-adjusted-methylation-assay system 106 continues to use the methylation-bias-adjustment-machine-learning model 424 to determine predicted bias scores for training datasets representing the different contextual sequences. In such subsequent training iterations, the bias-adjusted-methylation-assay system 106 further adjusts parameters of the methylation-bias-adjustment-machine-learning model 424 (e.g., by adjusting decision nodes or adding/removing decision trees) based on determined losses from comparisons of predicted bias scores and expected bias scores. In some cases, the bias-adjusted-methylation-assay system 106 performs training iterations until the parameters of the methylation-bias-adjustment-machine-learning model 404 do not change significantly across training iterations or otherwise satisfy a convergence criteria.

As indicated above, the bias-adjusted-methylation-assay system 106 can train a methylation-bias-adjustment-machine-learning model to determine bias scores directly or indirectly. In accordance with one or more embodiments, FIG. 4C depicts the bias-adjusted-methylation-assay system 106 training a methylation-bias-adjustment-machine-learning model to learn bias scores reflecting a dependency between specific contextual sequences and error rates of a given methylation assay in determining methylation-level values. To give context for that learned dependency, a dependency diagram 434 shows a contextual sequence 436a flanking a cytosine base at a CpG 1 site and a contextual sequence 436b flanking a cytosine base for a CpG 2 site. To illustrate the learned dependency, a bias-score graph 432 shows a bias score 1 for the contextual sequence 436a flanking the cytosine base at a CpG 1 site and a bias score 2 for the contextual sequence 43 6b flanking the cytosine base at a CpG 2 site.

In the bias-score graph 432, a vertical axis represents methylation-level values in terms of percentage of cytosines methylated. A horizontal axis represents different CpG sites, including CpG 1 site and CpG 2 site. As shown atop the vertical axis, the bias-adjusted-methylation-assay system 106 determines that expected methylation-level values are 100% for synthetically methylated cytosine bases within nucleotide sequences at each CpG site, including the CpG 1 site and the CpG 2 site. By contrast, the bias-adjusted-methylation-assay system 106 uses a given methylation assay to estimate 10% (or 0.10 value) of synthetically methylated cytosine bases are methylated-as an observed methylation-level value-at the CpG 1 site with a cytosine base flanked by the contextual sequence 436a. But the bias-adjusted-methylation-assay system 106 also uses the given methylation assay to estimate 90% (or 0.90 value) of synthetically methylated cytosine bases are methylated-as an observed methylation-level value-at the CpG 2 site with a cytosine base flanked by the contextual sequence 436b. Such incorrect observed methylation-level values reflect the vias of the given methylation assay.

According to the bias-score graph 432, therefore, the bias-adjusted-methylation-assay system 106 determines a bias score 1 at 90% (or 0.90) for the contextual sequence 436a based on a difference between the expected methylation-level value and the observed methylation-level value for the given methylation assay at the CpG 1 site. The bias-adjusted-methylation-assay system 106 also determines a bias score 2 at 10% (or 0.10) for the contextual sequence 436b based on a difference between the expected methylation-level value and the observed methylation-level value for the given methylation assay at the CpG 2 site. Consequently, the bias score 1 for the contextual sequence 436a corresponding to the CpG 1 site far exceeds the bias score 2 for the contextual sequence 436b corresponding to the CpG 2 site. As the difference between these bias scores illustrates, the bias of a given assay can differ significantly between specific contextual sequences that flank cytosine bases.

As further indicated by the dependency diagram 434 of FIG. 4C, in some embodiments, the bias-adjusted-methylation-assay system 106 trains a methylation-bias-adjustment-machine-learning model to learn a dependency between contextual sequences 436a and 436b and error rates of the given methylation assay in detecting or quantifying methylation levels at CpG sites corresponding to the contextual sequences 436a and 436b. By training a decision-tree model depicted in FIG. 4B, for instance, the bias-adjusted-methylation-assay system 106 trains the methylation-bias-adjustment-machine-learning model 424 to generate bias scores that directly reflect the dependency between specific contextual sequences and error rates of methylation-level values from the given methylation assay at CpG sites corresponding to the specific contextual sequences. By training a neural network depicted in FIG. 4A, the bias-adjusted-methylation-assay system 106 trains the methylation-bias-adjustment-machine-learning model 404 to generate predicted methylation-level values that indirectly reflect the dependency between specific contextual sequences and error rates of methylation-level values from the given methylation assay at CpG sites corresponding to the specific contextual sequences. In other words, after training, the predicted methylation-level values from the methylation-bias-adjustment-machine-learning model 404 reflect a learned dependency.

As indicated above, after training a methylation-bias-adjustment-machine-learning model, the bias-adjusted-methylation-assay system 106 can use a trained version of the methylation-bias-adjustment-machine-learning model to determine a predicted methylation-level value and/or a bias score for a specific contextual sequence flanking a target cytosine base. In some embodiments, the bias-adjusted-methylation-assay system 106 can likewise determine and visualize a contribution of different nucleobase classes at different positions within a contextual sequence based on predicted methylation-level values or bias scores output by a methylation-bias-adjustment-machine-learning model.

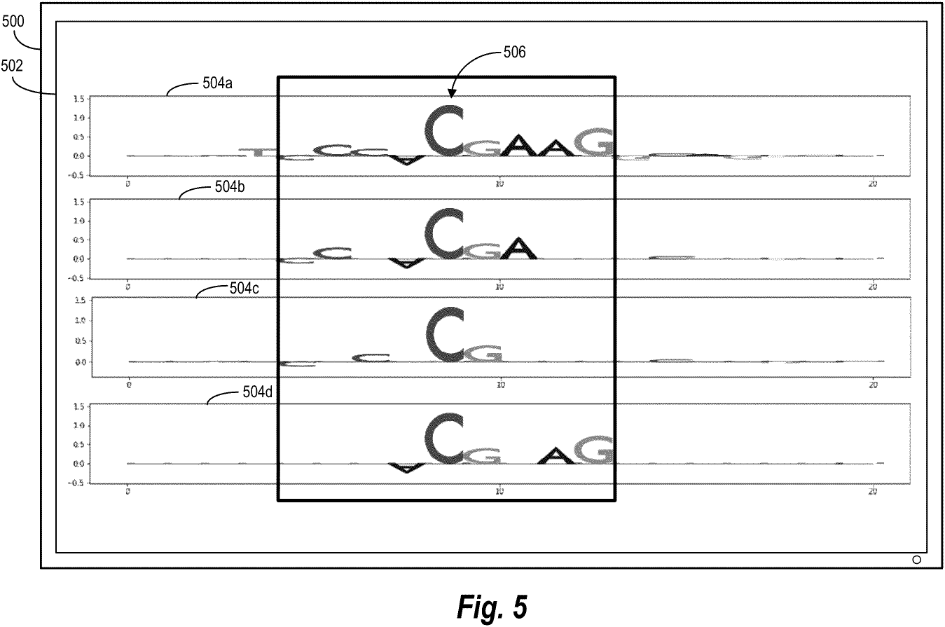

In accordance with one or more embodiments, FIG. 5 depicts the bias-adjusted-methylation-assay system 106 generating data for graphics indicating contribution metrics of different nucleobase classes at different contextual-sequence positions contributing to predicted methylation-level values. In particular, a computing device 500 presents, within a graphical user interface 502, nucleobase-class-contribution graphics 504a-504d comprising different sizes of nucleobase-letter images to indicate different contribution metrics of corresponding nucleobase classes when located at different positions within various contextual sequences. As explained below, the nucleobase-class-contribution graphics 504a-504d demonstrate that a particular nucleobase class (e.g., A, T, C, G) at a given contextual-sequence position can have varying positive or negative contributions to methylation-level values. While the bias-adjusted-methylation-assay system 106 comprises instructions that (upon execution) cause the computing device 500 to present graphics shown in FIG. 5, this disclosure will either refer to the computing device 500 of the bias-adjusted-methylation-assay system 106 as performing certain actions described below for simplicity without repeatedly describing such computer-executable instructions.

To determine contribution metrics for different nucleobase classes at different positions within contextual sequences, in some cases, the bias-adjusted-methylation-assay system 106 executes an Integrated Gradient (IG) algorithm. In particular, the bias-adjusted-methylation-assay system 106 can run an IG algorithm on data representing different contextual sequences input into a methylation-bias-adjustment-machine-learning model and corresponding predicted methylation-level values generated by the methylation-bias-adjustment-machine-learning model. By running an IG algorithm, for instance, the bias-adjusted-methylation-assay system 106 determines contribution metrics representing degrees to which different nucleobase classes contribute to predicted methylation-level values when the different nucleobase classes are located at specific positions within contextual sequences. After determining such contribution metrics, the bias-adjusted-methylation-assay system 106 generates data for the nucleobase-class-contribution graphics 504a-504d.

As shown by FIG. 5, the computing device 500 presents different sizes of nucleobase-letter images within the nucleobase-class-contribution graphics 504a-504d. At a center position 506 within each contextual sequence corresponding to each nucleobase-class-contribution graphic of FIG. 5, the computing device 500 presents a nucleobase-letter image for “C” identifying a cytosine base flanked by different contextual sequences. In this example, the contextual sequences depicted by nucleobase-letter images in each of the nucleobase-class-contribution graphics 504a-504d span nine nucleobases in length, where four nucleobases are positioned upstream from a cytosine base at a CpG site and four nucleobases are positioned downstream from the cytosine base. As further shown by the nucleobase-class-contribution graphics 504a-504d, a vertical axis for each of the nucleobase-class-contribution graphics 504a-504d represents different contribution metrics as determined by an IG algorithm, and a horizontal axis for each of the nucleobase-class-contribution graphics 504a-504d indicates positions of nucleobases within a contextual sequence.

As indicated by the nucleobase-letter images within the nucleobase-class-contribution graphics 504a-504d, the larger the nucleobase-letter image at a given contextual-sequence position, the larger positive or negative contribution metric for the corresponding nucleobase class at the given contextual-sequence position. Conversely, the smaller the nucleobase-letter image at a given contextual-sequence position, the smaller positive or negative contribution metric for the corresponding nucleobase class at the given contextual-sequence position.

As shown by the nucleobase-class-contribution graphics 504a and 504d, for instance, a “G” nucleobase-letter image-representing a guanine base at a fourth position downstream from a target cytosine base at a CpG site-is larger than other nucleobase-letter images above a zero contribution-metric value, thereby indicating that a guanine base at the fourth downstream position exhibits a larger positive contribution to predicted methylation-level values than other nucleobase classes within the contextual sequences depicted by the nucleobase-class-contribution graphics 504a and 504d. By contrast, as shown by the nucleobase-class-contribution graphics 504a, 504b, and 504c, an “A” nucleobase-letter image-representing an adenine base at a first position upstream from a target cytosine base at a CpG site-is larger than other nucleobase-letter images below a zero contribution-metric value, thereby indicating that an adenine base at the first upstream position exhibits a larger negative contribution to predicted methylation-level values than other nucleobase classes within the contextual sequences depicted by the nucleobase-class-contribution graphics 504a, 504b, and 504d.

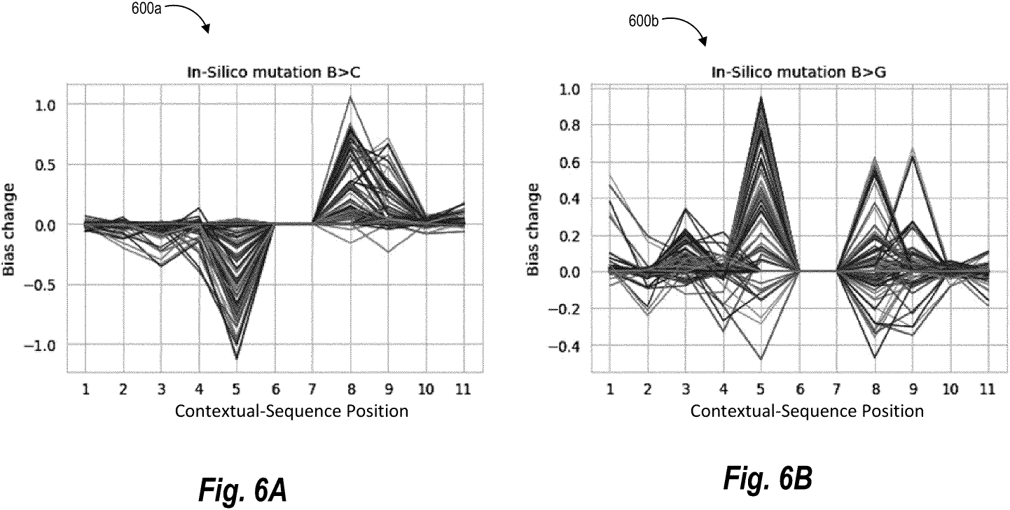

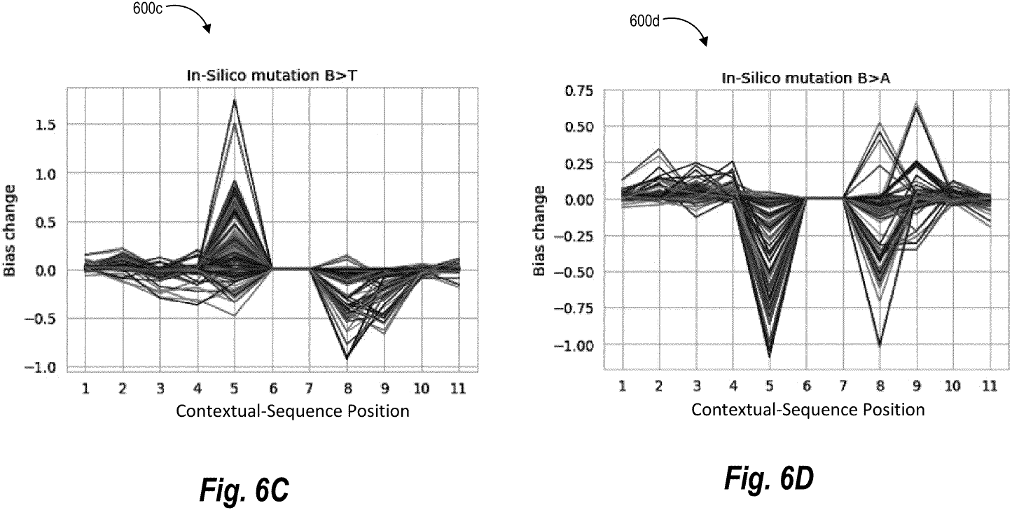

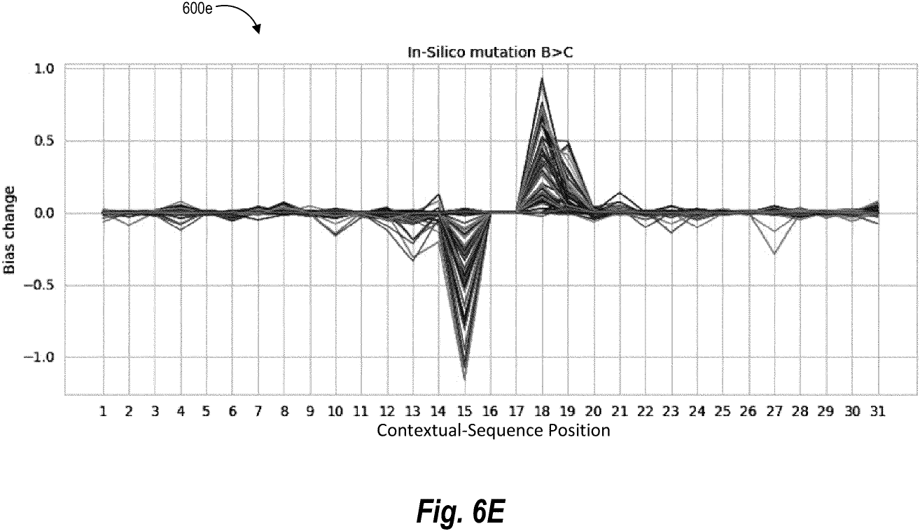

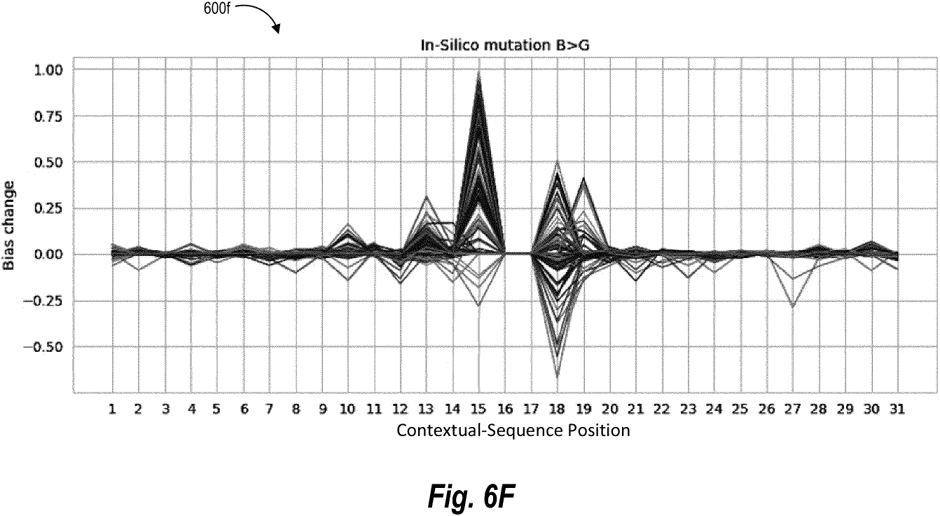

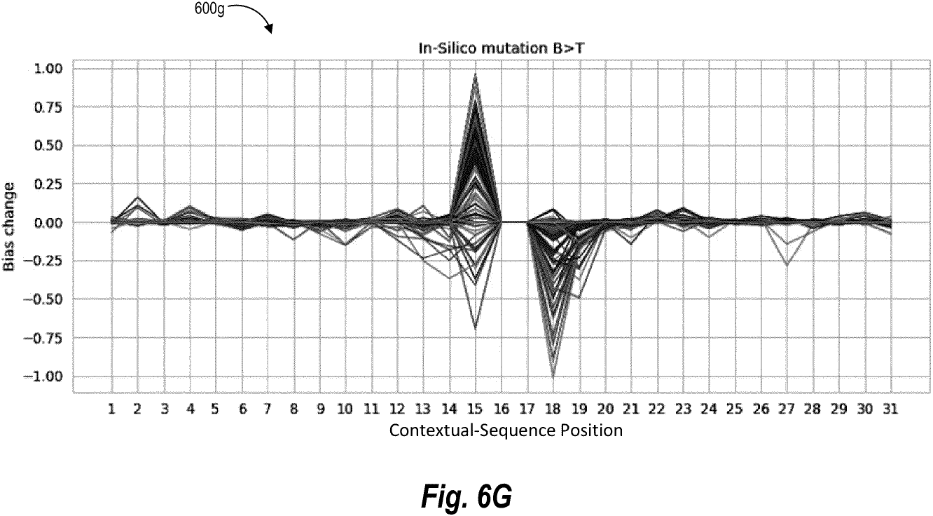

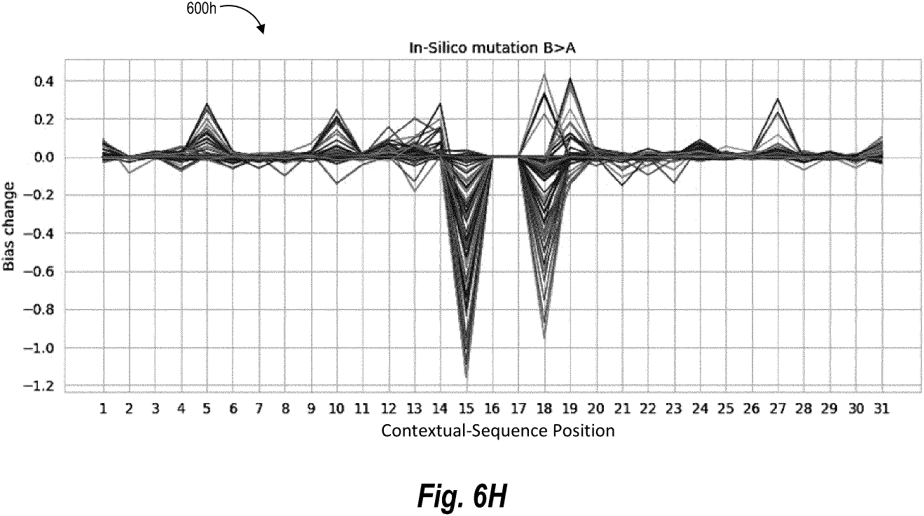

In addition to such nucleobase-class-contribution graphics, as indicated above, the bias-adjusted-methylation-assay system 106 can also generate data for graphics indicating degrees to which nucleobase-class changes at different positions within contextual sequences affect bias scores. In accordance with one or more embodiments, FIGS. 6A-6H each depict graphs showing a change in bias scores generated by a methylation-bias-adjustment-machine-learning model based on a change to a particular nucleobase class at various positions within a contextual sequence flanking a cytosine base. As exhibited by nucleobase-class-change graphs 600a-600h depicted in FIGS. 6A-6H, a change between nucleobase classes (e.g., A to C or T to G) at contextual-sequence positions closer to a target cytosine base have a relatively larger impact on bias scores than contextual-sequence positions further from the target cytosine base. While the bias-adjusted-methylation-assay system 106 (in some embodiments) comprises instructions that (upon execution) cause a computing device to present the nucleobase-class-change graphs 600a-600h in graphical user interfaces, this disclosure describes the nucleobase-class-change graphs 600a-600h for simplicity below without repeatedly describing such computer-executable instructions.

As shown in each of the nucleobase-class-change graphs 600a-600h of FIGS. 6A-6H, a vertical axis includes values representing a change in bias score, and a horizontal axis includes values representing a position within a contextual sequence. As shown in each of the nucleobase-class-change graphs 600a-600d of FIGS. 6A-6D, for instance, the cytosine base and guanine base that form a CpG site are represented at values 6 and 7 along the horizontal axis. As indicated by the values along the horizontal axis, the contextual sequences represented by the nucleobase-class-change graphs 600a-600d span eleven nucleobases with five nucleobases upstream and five nucleobases downstream from the target cytosine base at a CpG site.

By contrast, as shown in each of the nucleobase-class-change graphs 600e-600h of FIGS. 6E-6H, the cytosine base and guanine base that form a CpG site are located at values 16 and 17 along the horizontal axis. As indicated by the values along the horizontal axis, the contextual sequences represented by the nucleobase-class-change graphs 600e-600h span thirty-one nucleobases with fifteen nucleobases upstream and fifteen nucleobases downstream from the cytosine base at a CpG site.

As indicated by the nucleobase-class-change graphs 600a-600h of FIGS. 6A-6H, the bias-adjusted-methylation-assay system 106 use a methylation-bias-adjustment-machine-learning model to determine bias scores for contextual sequences in which a nucleobase class at each position of the contextual sequences was changed to a particular nucleobase class. In the nucleobase-class-change graph 600a of FIG. 6A, for instance, the bias-adjusted-methylation-assay system 106 (i) uses the methylation-bias-adjustment-machine-learning model to determine bias scores for different contextual sequences in which a given nucleobase (e.g., A, T, C, G) of the different contextual sequences was changed in silico to a particular nucleobase class of “C” for cytosine at each of nine different positions and (ii) determines bias-score changes when the given nucleobase class is changed to the particular nucleobase class at each position. The nucleobase-class-change graphs 600b, 600c, and 600d of FIGS. 6B, 6C, and 6D depict similar bias-score changes based on nucleobase-class changes from the given nucleobase class to G, T, and A, respectively, at different positions. Similarly, in the nucleobase-class-change graph 600e of FIG. 6E, the bias-adjusted-methylation-assay system 106 (i) uses the methylation-bias-adjustment-machine-learning model to determine bias scores for different contextual sequences in which a given nucleobase (e.g., A, T, C, G) of the different contextual sequences was changed in silico to a particular nucleobase class of “C” for cytosine at each of twenty-nine different positions and (ii) determines bias-score changes when the given nucleobase class is changed to the particular nucleobase class at each position. The nucleobase-class-change graphs 600f, 600g, and 600h of FIGS. 6F, 6G, and 6H depict similar bias-score changes based on nucleobase-class changes from the given nucleobase class to G, T, and A, respectively, at different positions.

As exhibited by the nucleobase-class-change graphs 600a-600d depicted in FIGS. 6A-6D, a change between nucleobase classes (e.g., A to C) at contextual-sequence positions that are closer to a target cytosine base have a relatively larger impact on bias-score changes than contextual-sequence positions that are further from a target cytosine base. In each of the nucleobase-class-change graphs 600a-600d, nucleobase-class changes at a first position upstream from the target cytosine base at a CpG site and a first position downstream from a guanine bases at the CpG site exhibit the largest bias-score changes in either a positive or negative direction. Extending contextual sequences from nine nucleobases flanking a CpG site to twenty-nine nucleobases flanking a CpG site does not materially change the impact of nucleobase-class changes at relative contextual-sequence positions. In each of the nucleobase-class-change graphs 600e-600h of FIGS. 6E-6H, nucleobase-class changes at a first position upstream from the target cytosine base at a CpG site and a first position downstream from a guanine bases at the CpG site likewise exhibit the largest bias-score changes in either a positive or negative direction.

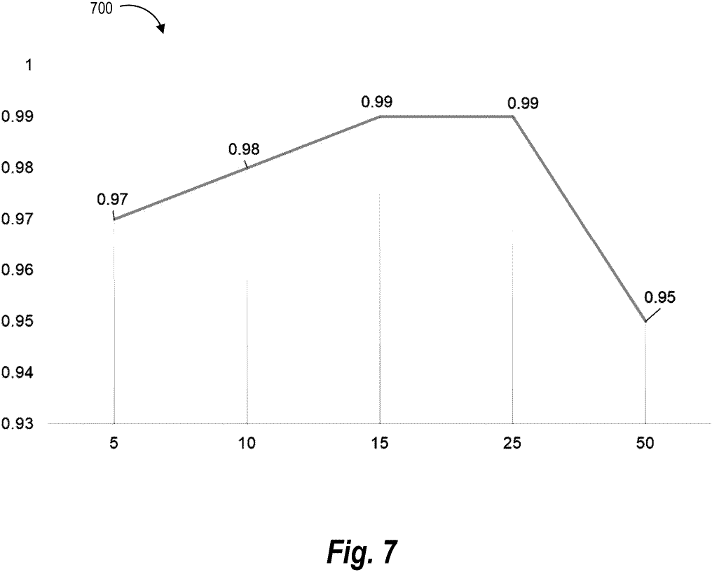

As indicated above, the bias-adjusted-methylation-assay system 106 can determine bias scores or predicted methylation-level values based on contextual sequences of different lengths (e.g., 5-50 upstream and 5-50 downstream nucleobases flanking a CpG site). To test the effect of contextual-sequence length on the accuracy of a CNN as a methylation-bias-adjustment-machine-learning model, researchers used a methylation-bias-adjustment-machine-learning model to generate predicted methylation-level values for contextual sequences of different nucleobase numbers and determined a percentage of CpG sites for which the predicted methylation-level values from the methylation-bias-adjustment-machine-learning model accurately represented or quantified methylation at the CpG sites. In accordance with one or more embodiments, FIG. 7 depicts a graph 700 showing a percentage of CpG sites for which the methylation-bias-adjustment-machine-learning model correctly determines predicted methylation-level values based on length of contextual sequence flanking a cytosine base at a CpG site.

As indicated by the vertical and horizontal axes, the graph 700 shows percentages of correct methylation-level values for contextual sequences comprising (i) five, ten, fifteen, twenty-five, or fifty upstream nucleobases flanking a cytosine base at a CpG site and (ii) five, ten, fifteen, twenty-five, or fifty downstream nucleobases, respectively, flanking the cytosine base at the CpG site. In other words, the horizontal axis of the graph 700 indicates the number of nucleobases flanking each of the upstream and downstream sides of the cytosine base at the CpG site.

As the graph 700 indicates, the methylation-bias-adjustment-machine-learning model generates predicted methylation-level values with approximately 99% accuracy based on contextual sequences comprising fifteen or twenty-five upstream nucleobases-and fifteen or twenty-five downstream nucleobases-flanking a target cytosine base at a CpG site. Accordingly, when implementing a customized CNN as a methylation-bias-adjustment-machine-learning model, the methylation-bias-adjustment-machine-learning model determines predicted methylation-level values with relatively highest accuracy by using contextual sequences with fifteen nucleobases flanking each side of a cytosine base at a CpG site-but does not increase the accuracy of methylation-level values when contextual sequences extend beyond fifteen nucleobases flanking each side.

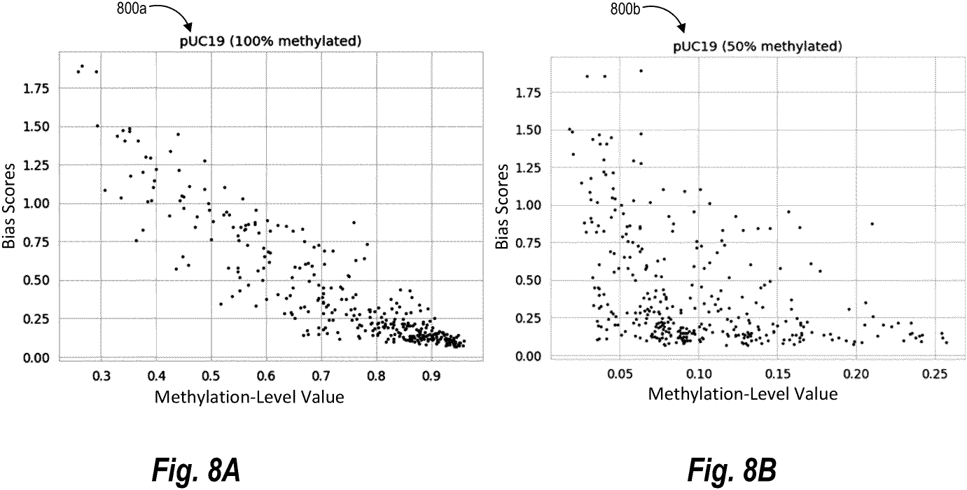

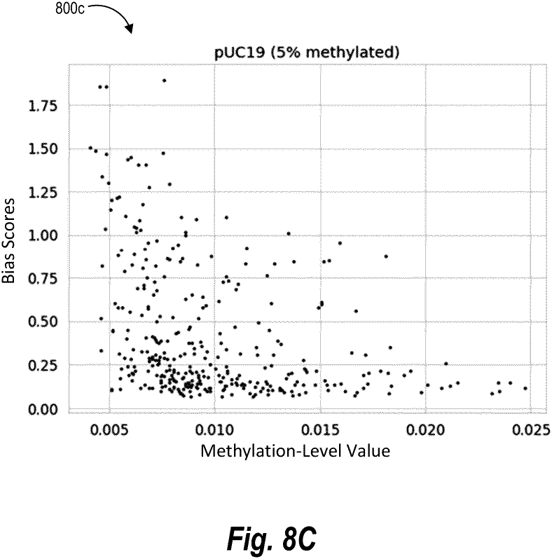

As indicated above, in some embodiments, the bias-adjusted-methylation-assay system 106 synthetically methylates cytosines within nucleotide sequences and trains a methylation-bias-adjustment-machine-learning model to learn dependencies between different contextual sequences flanking such cytosines and a bias (or error) with which a given methylation assay determines methylation-level values for the synthetically methylated cytosines. But the bias-adjusted-methylation-assay system 106 can use other methylation experiments to train a methylation-bias-adjustment-machine-learning model to learn such dependencies. In accordance with one or more embodiments, FIGS. 8A-8C depict graphs 800a-800c showing methylation-level values (e.g., beta values) from a given methylation assay and corresponding bias scores determined by a methylation-bias-adjustment-machine-learning model. As depicted by FIGS. 8A-8C, the graphs 800a, 800b, and 800c depict such methylation-level values and corresponding bias scores learned by a methylation-bias-adjustment-machine-learning model using plasmids comprising cytosine bases synthetically methylated at approximately 100%, 50%, and 5%, respectively.

As indicated by the graphs 800a-800c, the methylation-bias-adjustment-machine-learning model can learn dependencies between bias scores and methylation-level values from a given methylation assay effectively independent of the amount or percentage of synthetically methylated cytosines. In each of the graphs 800a-800c, the methylation-bias-adjustment-machine-learning model determines bias scores with approximately a same learned dependency or correlation on corresponding methylation-level values determined by the given methylation assay.

When the cytosine bases of plasmids are synthetically methylated at approximately 100%, as depicted by the graph 800a of FIG. 8A, the methylation-bias-adjustment-machine-learning model learns a correlation or dependency between a methylation-level value of approximately 0.90 and a bias score of approximately 0.10. When the cytosine bases of plasmids are synthetically methylated at approximately 50%, as depicted by the graph 800b of FIG. 8B, the methylation-bias-adjustment-machine-learning model learns a correlation or dependency between a methylation-level value of approximately 0.25 and a bias score of approximately 0.10. While the graphs 800b and 800c for synthetically methylated cytosines of approximately 50% and 5% do not depict a dependency between methylation-level values and bias scores as clearly or demonstrably as the graph 800a for synthetically methylated cytosines of approximately 100%, the methylation-bias-adjustment-machine-learning model can nevertheless learn a similar dependency when using different approaches to measuring bias of the given methylation assay.

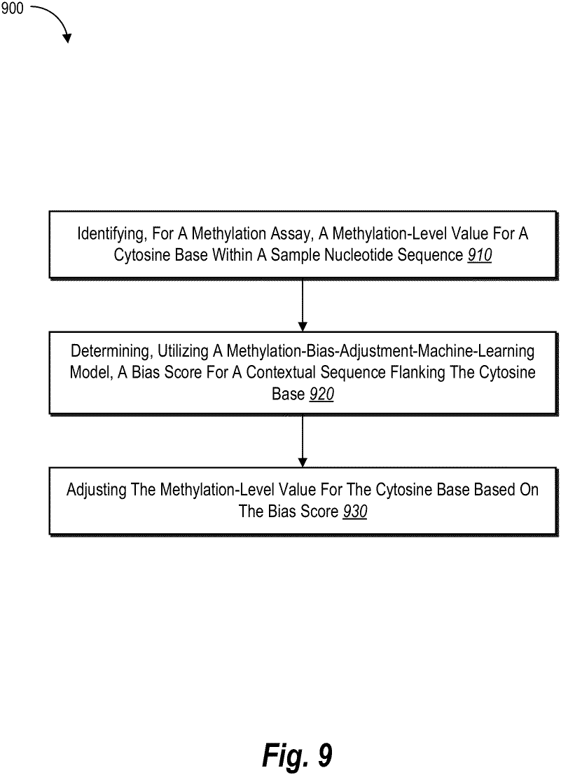

Turning now to FIG. 9, this figure illustrates a flowchart of a series of acts 900 of utilizing a machine-learning model to determine bias scores of a methylation assay for a specific contextual sequence of a target cytosine base and adjusting a methylation-level value for the target cytosine base from the methylation assay in accordance with one or more embodiments of the present disclosure. While FIG. 9 illustrates acts according to one embodiment, alternative embodiments may omit, add to, reorder, and/or modify any of the acts shown in FIG. 9. The acts of FIG. 9 can be performed as part of a method. Alternatively, a non-transitory computer readable storage medium can comprise instructions that, when executed by one or more processors, cause a computing device or a system to perform the acts depicted in FIG. 9. In still further embodiments, a system comprising at least one processor and a non-transitory computer readable medium comprising instructions that, when executed by one or more processors, cause the system to perform the acts of FIG. 9.

As shown in FIG. 9, the acts 900 include an act 910 of identifying, for a methylation assay, a methylation-level value for a cytosine base within a sample nucleotide sequence. In particular, in some embodiments, the act 910 includes identifying, for a methylation assay, a methylation-level value indicating a level of methylation of a cytosine base within a sample nucleotide sequence. Additionally, or alternatively, the act 910 includes identifying, for a methylation assay, methylation-level values indicating levels of methylation of cytosine bases within one or more sample nucleotide sequences. As indicated above, in some embodiments, identifying the methylation-level value comprises identifying a beta value or an M value for the cytosine base within the sample nucleotide sequence.

As indicated above, in some cases, identifying the methylation-level value comprises accessing or receiving, from a computing device, the methylation-level value determined by the methylation assay. Additionally, in certain embodiments, identifying the methylation-level value comprises determining, utilizing a methylation assay, a methylation-level value indicating a level of methylation of a cytosine base within a sample nucleotide sequence. Additionally, or alternatively, identifying the methylation-level value comprises includes determining, utilizing a methylation assay, methylation-level values indicating levels of methylation of cytosine bases within one or more sample nucleotide sequences. As indicated above, in some embodiments, determining the methylation-level value comprises determining a beta value or an M value for the cytosine base within the sample nucleotide sequence.

As further shown in FIG. 9, the acts 900 include an act 920 of determining, utilizing a methylation-bias-adjustment-machine-learning model, a bias score for a contextual sequence flanking the cytosine base. In particular, in some embodiments, the act 920 includes determining, utilizing a methylation-bias-adjustment-machine-learning model, bias scores for contextual sequences flanking respective cytosine bases within the one or more sample nucleotide sequences. In some cases, the methylation-bias-adjustment-machine-learning model comprises a neural network or one or more decision trees.

As suggested above, in certain embodiments, determining the bias score comprises determining a score indicating a degree to which the methylation assay errs in detecting methylation of the cytosine base when flanked by the contextual sequence. As further suggested above, in some cases, the contextual sequence comprises a threshold number of nucleobases upstream from the cytosine base and a threshold number of nucleobases downstream from the cytosine base. Relatedly, in some cases, determining, utilizing the methylation-bias-adjustment-machine-learning model, the bias scores for the contextual sequences comprises: determining a first bias score for a first contextual sequence comprising a threshold number of nucleobases upstream from a first cytosine base and a threshold number of nucleobases downstream from the first cytosine base; and determining a second bias score for a second contextual sequence comprising the threshold number of nucleobases upstream from a second cytosine base and the threshold number of nucleobases downstream from the second cytosine base.

Relatedly, in certain implementations, determining, utilizing the methylation-bias-adjustment-machine-learning model, the bias score for the contextual sequence comprises generating, from the methylation-bias-adjustment-machine-learning model, the bias score based on a dataset representing the contextual sequence. By contrast, in certain cases, determining, utilizing the methylation-bias-adjustment-machine-learning model, the bias score for the contextual sequence comprises: determining an expected methylation-level value for the cytosine base flanked by the contextual sequence within a synthetically methylated nucleotide sequence; generating, from the methylation-bias-adjustment-machine-learning model, a predicted methylation-level value based on a dataset representing the contextual sequence; and determining, as the bias score for the contextual sequence, a value difference between the predicted methylation-level value from the methylation-bias-adjustment-machine-learning model and the expected methylation-level value for the cytosine base flanked by the contextual sequence.

Alternatively, in some cases, determining, utilizing the methylation-bias-adjustment-machine-learning model, the bias score for the contextual sequence comprises: generating, from the methylation-bias-adjustment-machine-learning model, a predicted methylation-level value based on a dataset representing the contextual sequence; and determining, as the bias score for the contextual sequence, a value difference between the predicted methylation-level value from the methylation-bias-adjustment-machine-learning model and the methylation-level value from the methylation assay.

As indicated above, in some cases, determining, utilizing the methylation-bias-adjustment-machine-learning model, the bias scores for the contextual sequences comprises: generating, from the methylation-bias-adjustment-machine-learning model, a first bias score based on a first contextual sequence; and generating, from the methylation-bias-adjustment-machine-learning model, a second bias score based on a second contextual sequence. By contrast, in certain implementations, determining, utilizing the methylation-bias-adjustment-machine-learning model, the bias scores for the contextual sequences comprises: determining a first expected methylation-level value for a first cytosine base flanked by a first contextual sequence within a synthetically methylated nucleotide sequence and a second expected methylation-level value for a second cytosine base flanked by a second contextual sequence within the synthetically methylated nucleotide sequence; generating, from the methylation-bias-adjustment-machine-learning model, a first predicted methylation-level value and a second predicted methylation-level value respectively based on a first dataset representing the first contextual sequence flanking the first cytosine base and a second dataset representing the second contextual sequence flanking the second cytosine base; determining, for the first contextual sequence, a first bias score as a first value difference between the first predicted methylation-level value from the methylation-bias-adjustment-machine-learning model and the first expected methylation-level value for the first cytosine base flanked by the first contextual sequence; and determining, for the second contextual sequence, a second bias score as a second value difference between the second predicted methylation-level value from the methylation-bias-adjustment-machine-learning model and the second expected methylation-level value for the second cytosine base flanked by the second contextual sequence.

Alternatively, in some cases, determining, utilizing the methylation-bias-adjustment-machine-learning model, the bias scores for the contextual sequences comprises: generating, from the methylation-bias-adjustment-machine-learning model, a first predicted methylation-level value and a second predicted methylation-level value respectively based on a first dataset representing a first contextual sequence flanking a first cytosine base and a second dataset representing a second contextual sequence flanking a second cytosine base; determining, for the first contextual sequence, a first bias score as a first value difference between the first predicted methylation-level value from the methylation-bias-adjustment-machine-learning model and a first methylation-level value from the methylation assay; and determining, for the second contextual sequence, a second bias score as a second value difference between the second predicted methylation-level value from the methylation-bias-adjustment-machine-learning model and a second methylation-level value from the methylation assay.

As further shown in FIG. 9, the acts 900 include an act 930 of adjusting the methylation-level value for the cytosine base based on the bias score. In particular, in certain implementations, the act 930 includes adjusting the methylation-level value for the cytosine base based on the bias score for the contextual sequence. As suggested above, in some embodiments, the act 930 includes adjusting one or more of the methylation-level values for one or more of the cytosine bases based on one or more of the bias scores. As further suggested above, in some cases, the act 930 includes adjusting one or more of the methylation-level values for one or more of the cytosine bases corresponding to one or more genomic coordinates for a promoter region or a gene associated with a disease.

In addition to the acts 910-930, in certain implementations, the acts 900 further include identifying, based on the bias score, a genomic coordinate for the cytosine base subject to a bias of the methylation assay. Similarly, in certain cases, the acts 900 include identifying, based on one or more of the bias scores, a genomic region for one or more cytosine bases of the cytosine bases subject to a bias of the methylation assay.

As suggested above, in addition or in the alternative, in some embodiments, the acts 900 include providing, for display within a graphical user interface, a graphic indicating a degree to which a nucleobase-class change at one or more positions within contextual sequences affects bias scores. Similarly, in some cases, the acts 900 include providing, for display within a graphical user interface, a graphic indicating a degree to which nucleobase-class changes at different positions within the contextual sequences affect the bias scores.

Further, in certain implementations, the acts 900 include providing, for display within a graphical user interface, a graphic indicating a contribution metric for a nucleobase class at one or more positions within contextual sequences contributing to predicted methylation-level values. Similarly, in some cases, the acts 900 include providing, for display within a graphical user interface, a graphic indicating contribution metrics for different nucleobase classes at different positions within the contextual sequences contributing to predicted methylation-level values.

Additionally, or alternatively, in some cases, the acts 900 include determining, utilizing an additional methylation-bias-adjustment-machine-learning model, an additional bias score for the contextual sequence flanking the cytosine base; determining a composite bias score for the contextual sequence based on the bias score and the additional bias score; and adjusting the methylation-level value for the cytosine base based on the composite bias score.

Similarly, in certain implementations, the acts 900 include determining, utilizing an additional methylation-bias-adjustment-machine-learning model, additional bias scores for the contextual sequences flanking the cytosine bases; determining composite bias scores for the contextual sequences based on the bias scores and the additional bias scores; and adjusting one or more of the methylation-level values for one or more of the cytosine bases based on respective composite bias scores.

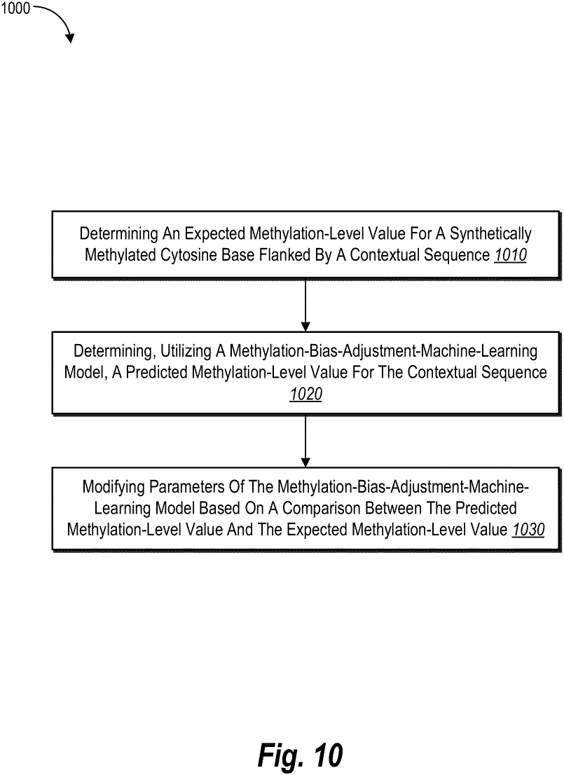

Turning now to FIG. 10, this figure illustrates a flowchart of a series of acts 1000 of training a methylation-bias-adjustment-machine-learning model to determine predicted methylation-level values for specific contextual sequences in accordance with one or more embodiments of the present disclosure. While FIG. 10 illustrates acts according to one embodiment, alternative embodiments may omit, add to, reorder, and/or modify any of the acts shown in FIG. 10. The acts of FIG. 10 can be performed as part of a method. Alternatively, a non-transitory computer readable storage medium can comprise instructions that, when executed by one or more processors, cause a computing device or a system to perform the acts depicted in FIG. 10. In still further embodiments, a system comprising at least one processor and a non-transitory computer readable medium comprising instructions that, when executed by one or more processors, cause the system to perform the acts of FIG. 10.

As shown in FIG. 10, the acts 1000 include an act 1010 of determining an expected methylation-level value for a synthetically methylated cytosine base flanked by a contextual sequence. In particular, in some embodiments, the act 1010 includes determining expected methylation-level values indicating levels of methylation of synthetically methylated cytosine bases flanked by respective contextual sequences.

As further shown in FIG. 10, the acts 1000 include an act 1020 of determining, utilizing a methylation-bias-adjustment-machine-learning model, a predicted methylation-level value for the contextual sequence. In particular, in some embodiments, the act 1020 includes determining the predicted methylation-level value by determining, utilizing the methylation-bias-adjustment-machine-learning model across training iterations, predicted methylation-level values for the respective contextual sequences.

As further shown in FIG. 10, the acts 1000 include an act 1030 of modifying one or more parameters of the methylation-bias-adjustment-machine-learning model based on a comparison between the predicted methylation-level value and the expected methylation-level value for the contextual sequence. In particular, in certain implementations, the act 1030 includes modifying one or more parameters of the methylation-bias-adjustment-machine-learning model based on comparisons across the training iterations between individual predicted methylation-level values and individual expected methylation-level values for the respective contextual sequences.

Further, in some cases, modifying the one or more parameters of the methylation-bias-adjustment-machine-learning model based on the comparison between the predicted methylation-level value and the expected methylation-level value comprises: determining a training value difference between the predicted methylation-level value and the expected methylation-level value; and modifying one or more weights of the methylation-bias-adjustment-machine-learning model based on the training value difference.

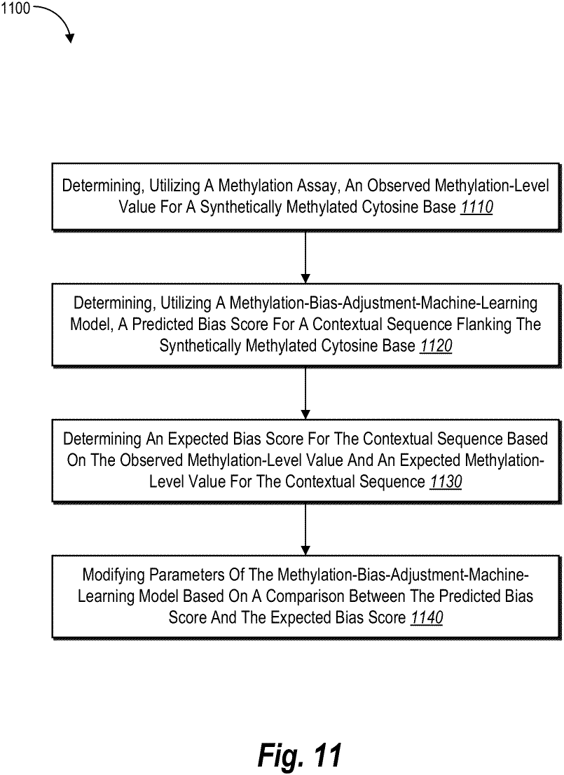

Turning now to FIG. 11, this figure illustrates a flowchart of a series of acts 1100 of training a methylation-bias-adjustment-machine-learning model to determine bias scores for specific contextual sequences in accordance with one or more embodiments of the present disclosure. While FIG. 11 illustrates acts according to one embodiment, alternative embodiments may omit, add to, reorder, and/or modify any of the acts shown in FIG. 11. The acts of FIG. 11 can be performed as part of a method. Alternatively, a non-transitory computer readable storage medium can comprise instructions that, when executed by one or more processors, cause a computing device or a system to perform the acts depicted in FIG. 11. In still further embodiments, a system comprising at least one processor and a non-transitory computer readable medium comprising instructions that, when executed by one or more processors, cause the system to perform the acts of FIG. 11.

As shown in FIG. 11, the acts 1100 include an act 1110 of determining, utilizing a methylation assay, an observed methylation-level value indicating a level of methylation of a synthetically methylated cytosine base. In some cases, the act 1110 comprises determining, utilizing a methylation assay, an observed methylation-level value indicating a level of methylation of a synthetically methylated cytosine base within a nucleotide sequence. For example, in some embodiments, the act 1110 includes determining observed methylation-level values indicating levels of methylation of synthetically methylated cytosine bases within one or more nucleotide sequences.

As further shown in FIG. 11, the acts 1100 include an act 1120 of determining, utilizing a methylation-bias-adjustment-machine-learning model, a predicted bias score for a contextual sequence flanking the synthetically methylated cytosine base. In particular, in some embodiments, the act 1120 includes determining the predicted bias score comprises determining, utilizing the methylation-bias-adjustment-machine-learning model across training iterations, predicted bias scores for contextual sequences respectively flanking the synthetically methylated cytosine bases.

As further shown in FIG. 11, the acts 1100 include an act 1130 of determining an expected bias score for the contextual sequence based on a comparison of the observed methylation-level value and an expected methylation-level value for the contextual sequence. In particular, in certain implementations, the act 1130 includes determining expected bias scores for the contextual sequences based on comparisons across the training iterations of individual observed methylation-level values and individual expected methylation-level values for respective contextual sequences.

As further shown in FIG. 11, the acts 1100 include an act 1140 of modifying one or more parameters of the methylation-bias-adjustment-machine-learning model based on a comparison between the predicted bias score and the expected bias score for the contextual sequence. In particular, in certain implementations, the act 1140 includes modifying the one or more parameters comprises modifying parameters of the methylation-bias-adjustment-machine-learning model based on comparisons across the training iterations of individual predicted bias scores and individual expected bias scores for the respective contextual sequences.

Further, in some cases, modifying the one or more parameters of the methylation-bias-adjustment-machine-learning model based on the comparison of the predicted bias score and the expected bias score comprises: determining a training score difference between the predicted bias score and the expected bias score; and modifying at least a parameter for one or more decision trees of the methylation-bias-adjustment-machine-learning model based on the training score difference.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. No. 6,210,891; U.S. Pat. No. 6,258,568 and U.S. Pat. No. 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. No. 7,427,673, and U.S. Pat. No. 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Pat. Application Publication No. 2012/0270305 and U.S. Pat. Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Pat. Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Pat. Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. No. 6,969,488, U.S. Pat. No. 6,172,218, and U.S. Pat. No. 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. No. 7,329,492 and U.S. Pat. No. 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Pat. Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and US Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeqTM platform (Illumina, Inc., San Diego, CA) and devices described in US Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture microdissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the bias-adjusted-methylation-assay system 106 can include software, hardware, or both. For example, the components of the bias-adjusted-methylation-assay system 106 can include one or more instructions stored on a computer-readable storage medium and executable by processors of one or more computing devices (e.g., the user client device 110). When executed by the one or more processors, the computer-executable instructions of the bias-adjusted-methylation-assay system 106 can cause the computing devices to perform the bubble detection methods described herein. Alternatively, the components of the bias-adjusted-methylation-assay system 106 can comprise hardware, such as special purpose processing devices to perform a certain function or group of functions. Additionally, or alternatively, the components of the bias-adjusted-methylation-assay system 106 can include a combination of computer-executable instructions and hardware.

Furthermore, the components of the bias-adjusted-methylation-assay system 106 performing the functions described herein with respect to the bias-adjusted-methylation-assay system 106 may, for example, be implemented as part of a stand-alone application, as a module of an application, as a plug-in for applications, as a library function or functions that may be called by other applications, and/or as a cloud-computing model. Thus, components of the bias-adj usted-methylation-assay system 106 may be implemented as part of a stand-alone application on a personal computing device or a mobile device. Additionally, or alternatively, the components of the bias-adjusted-methylation-assay system 106 may be implemented in any application that provides sequencing services including, but not limited to Illumina BaseSpace, BeadArray, BeadChip, Illumina DRAGEN, Infinium Methylation Assay, or Illumina TruSight software. “Illumina,” “BeadArray,” “BeadChip,” “BaseSpace,” “DRAGEN,” “Infinium Methylation Assay,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multiprocessor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 12 illustrates a block diagram of a computing device 1200 that may be configured to perform one or more of the processes described above. One will appreciate that one or more computing devices such as the computing device 1200 may implement the bias-adjusted-methylation-assay system 106 and the sequencing system 104. As shown by FIG. 12, the computing device 1200 can comprise a processor 1202, a memory 1204, a storage device 1206, an I/O interface 1208, and a communication interface 1210, which may be communicatively coupled by way of a communication infrastructure 1212. In certain embodiments, the computing device 1200 can include fewer or more components than those shown in FIG. 12. The following paragraphs describe components of the computing device 1200 shown in FIG. 12 in additional detail.

In one or more embodiments, the processor 1202 includes hardware for executing instructions, such as those making up a computer program. As an example, and not by way of limitation, to execute instructions for dynamically modifying workflows, the processor 1202 may retrieve (or fetch) the instructions from an internal register, an internal cache, the memory 1204, or the storage device 1206 and decode and execute them. The memory 1204 may be a volatile or non-volatile memory used for storing data, metadata, and programs for execution by the processor(s). The storage device 1206 includes storage, such as a hard disk, flash disk drive, or other digital storage device, for storing data or instructions for performing the methods described herein.

The I/O interface 1208 allows a user to provide input to, receive output from, and otherwise transfer data to and receive data from computing device 1200. The I/O interface 1208 may include a mouse, a keypad or a keyboard, a touch screen, a camera, an optical scanner, network interface, modem, other known I/O devices or a combination of such I/O interfaces. The I/O interface 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1208 is configured to provide graphical data to a display for presentation to a user. The graphical data may be representative of one or more graphical user interfaces and/or any other graphical content as may serve a particular implementation.

The communication interface 1210 can include hardware, software, or both. In any event, the communication interface 1210 can provide one or more interfaces for communication (such as, for example, packet-based communication) between the computing device 1200 and one or more other computing devices or networks. As an example, and not by way of limitation, the communication interface 1210 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1210 may facilitate communications with various types of wired or wireless networks. The communication interface 1210 may also facilitate communications using various communication protocols. The communication infrastructure 1212 may also include hardware, software, or both that couples components of the computing device 1200 to each other. For example, the communication interface 1210 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A method comprising:
identifying, for a methylation assay, a methylation-level value indicating a level of methylation of a cytosine base within a sample nucleotide sequence;
determining, utilizing a methylation-bias-adjustment-machine-learning model, a bias score for a contextual sequence flanking the cytosine base; and
adjusting the methylation-level value for the cytosine base based on the bias score for the contextual sequence.

2. The method of claim 1, further comprising identifying, based on the bias score, a genomic coordinate for the cytosine base subject to a bias of the methylation assay.

3. The method of claim 1, wherein identifying the methylation-level value comprises identifying a beta value or an M value for the cytosine base within the sample nucleotide sequence.

4. The method of claim 1, wherein determining the bias score comprises determining a score indicating a degree to which the methylation assay errs in detecting methylation of the cytosine base when flanked by the contextual sequence.

5. The method of claim 1, wherein determining, utilizing the methylation-bias-adjustment-machine-learning model, the bias score for the contextual sequence comprises:
determining an expected methylation-level value for the cytosine base flanked by the contextual sequence within a synthetically methylated nucleotide sequence;
generating, from the methylation-bias-adjustment-machine-learning model, a predicted methylation-level value based on a dataset representing the contextual sequence; and
determining, as the bias score for the contextual sequence, a value difference between the predicted methylation-level value from the methylation-bias-adjustment-machine-learning model and the expected methylation-level value for the cytosine base flanked by the contextual sequence.

6. The method of claim 1, wherein determining, utilizing the methylation-bias-adjustment-machine-learning model, the bias score for the contextual sequence comprises generating, from the methylation-bias-adjustment-machine-learning model, the bias score based on a dataset representing the contextual sequence.

7. The method of claim 1, wherein determining the bias score for the contextual sequence comprising a threshold number of nucleobases upstream from the cytosine base and a threshold number of nucleobases downstream from the cytosine base.

8. The method of claim 1, further comprising:
determining, utilizing an additional methylation-bias-adjustment-machine-learning model, an additional bias score for the contextual sequence flanking the cytosine base;
determining a composite bias score for the contextual sequence based on the bias score and the additional bias score; and
adjusting the methylation-level value for the cytosine base based on the composite bias score.

9. A system comprising:
at least one processor; and
a non-transitory computer readable medium comprising instructions that, when executed by the at least one processor, cause the system to:
identify, for a methylation assay, methylation-level values indicating levels of methylation of cytosine bases within one or more sample nucleotide sequences;
determine, utilizing a methylation-bias-adjustment-machine-learning model, bias scores for contextual sequences flanking respective cytosine bases within the one or more sample nucleotide sequences; and
adjust one or more of the methylation-level values for one or more of the cytosine bases based on one or more of the bias scores.

10. The system of claim 9, further comprising instructions that, when executed by the at least one processor, cause the system to identify, based on one or more of the bias scores, a genomic region for one or more cytosine bases of the cytosine bases subject to a bias of the methylation assay.

11. The system of claim 9, further comprising instructions that, when executed by the at least one processor, cause the system to adjust one or more of the methylation-level values for one or more of the cytosine bases corresponding to one or more genomic coordinates for a promoter region or a gene associated with a disease.

12. The system of claim 9, further comprising instructions that, when executed by the at least one processor, cause the system to determine, utilizing the methylation-bias-adjustment-machine-learning model, the bias scores for the contextual sequences by:
generating, from the methylation-bias-adjustment-machine-learning model, a first bias score based on a first contextual sequence; and
generating, from the methylation-bias-adjustment-machine-learning model, a second bias score based on a second contextual sequence.

13. The system of claim 9, further comprising instructions that, when executed by the at least one processor, cause the system to determine, utilizing the methylation-bias-adjustment-machine-learning model, the bias scores for the contextual sequences by:
determining a first expected methylation-level value for a first cytosine base flanked by a first contextual sequence within a synthetically methylated nucleotide sequence and a second expected methylation-level value for a second cytosine base flanked by a second contextual sequence within the synthetically methylated nucleotide sequence;
generating, from the methylation-bias-adjustment-machine-learning model, a first predicted methylation-level value and a second predicted methylation-level value respectively based on a first dataset representing the first contextual sequence flanking the first cytosine base and a second dataset representing the second contextual sequence flanking the second cytosine base;
determining, for the first contextual sequence, a first bias score as a first value difference between the first predicted methylation-level value from the methylation-bias-adjustment-machine-learning model and the first expected methylation-level value for the first cytosine base flanked by the first contextual sequence; and
determining, for the second contextual sequence, a second bias score as a second value difference between the second predicted methylation-level value from the methylation-bias-adjustment-machine-learning model and the second expected methylation-level value for the second cytosine base flanked by the second contextual sequence.

14. The system of claim 9, further comprising instructions that, when executed by the at least one processor, cause the system to determine, utilizing the methylation-bias-adjustment-machine-learning model, the bias scores for the contextual sequences by:
determining a first bias score for a first contextual sequence comprising a threshold number of nucleobases upstream from a first cytosine base and a threshold number of nucleobases downstream from the first cytosine base; and
determining a second bias score for a second contextual sequence comprising the threshold number of nucleobases upstream from a second cytosine base and the threshold number of nucleobases downstream from the second cytosine base.

15. The system of claim 9, further comprising instructions that, when executed by the at least one processor, cause the system to:
determine, utilizing an additional methylation-bias-adjustment-machine-learning model, additional bias scores for the contextual sequences flanking the cytosine bases;
determine composite bias scores for the contextual sequences based on the bias scores and the additional bias scores; and
adjust one or more of the methylation-level values for one or more of the cytosine bases based on respective composite bias scores.

16. The system of claim 9, further comprising instructions that, when executed by the at least one processor, cause the system to provide, for display within a graphical user interface, a graphic indicating a degree to which nucleobase-class changes at different positions within the contextual sequences affect the bias scores.

17. A non-transitory computer readable medium comprising instructions that, when executed by at least one processor, cause a system to:
identify, for a methylation assay, a methylation-level value indicating a level of methylation of a cytosine base within a sample nucleotide sequence;
determine, utilizing a methylation-bias-adjustment-machine-learning model, a bias score for a contextual sequence flanking the cytosine base; and
adjust the methylation-level value for the cytosine base based on the bias score for the contextual sequence.

18. The non-transitory computer readable medium of claim 17, wherein the methylation-bias-adjustment-machine-learning model comprises a neural network or one or more decision trees.

19. The non-transitory computer readable medium of claim 17, further comprising instructions that, when executed by the at least one processor, cause the system to provide, for display within a graphical user interface, a graphic indicating a degree to which a nucleobase-class change at one or more positions within contextual sequences affects bias scores.

20. The non-transitory computer readable medium of claim 17, further comprising instructions that, when executed by the at least one processor, cause the system to provide, for display within a graphical user interface, a graphic indicating a contribution metric for a nucleobase class at one or more positions within contextual sequences contributing to predicted methylation-level values.
